(19) United States
(12) Patent Application Publication
(10) Pub. No.: US 2022/0047472 A1
(43) Pub. Date: Feb. 17, 2022

(54) MICROEMULSIONS AND METHODS OF USE

(71) Applicant: MOROCCANOIL ISRAEL, LTD., Rishon Lezion (IL)

(72) Inventors: Tycho J. Speaker, Santa Cruz, CA (US); Lavinia Codruta Popescu, New York, NY (US)

(73) Assignee: MOROCCANOIL ISRAEL, LTD., Rishon Lezion (IL)

(21) Appl. No.: 17/278,918

(22) PCT Filed: Sep. 25, 2019
(86) PCT No.: PCT/IB2019/058108

§ 371 Date: Mar. 23, 2021

Related U.S. Application Data
(60) Provisional application No. 62/736,274, filed on Sep. 25, 2018.

(51) Int. Cl. A61K 8/06; A61K 8/37; A61K 8/92; A61K 8/73; A61Q 5/00; D06B 1/00

(52) U.S. Cl. CPC A61K 8/068; A61K 8/375; A61K 8/922; A61Q 9/02; A61K 8/736; A61Q 5/002; D06B 1/00; A61K 8/733

(57) ABSTRACT

Compositions are described that include an oil, a surfactant, and optionally a charged polymer complex, where the composition may also include an aqueous phase to provide a microemulsion. Methods of manufacturing the compositions are also described, along with methods of using the compositions in home care and personal care.

### CROSS-REFERENCE TO RELATED APPLICATIONS

This application claims priority under 35 U.S.C. § 119(e) to U.S. Provisional Application No. 62/736,274 filed Sep. 25, 2018, the content of which is hereby incorporated by reference in its entirety.

### FIELD

The present invention is directed to compositions including microemulsions and methods of making and using such compositions.

### BACKGROUND

The following discussion is provided to aid the reader in understanding the disclosure and is not admitted to describe or constitute prior art thereto.

An emulsion is a fluid system in which liquid droplets are dispersed in a liquid. The droplets may be amorphous, liquid-crystalline, or any mixture thereof. The diameters of the droplets constituting the dispersed phase range from approximately 10 nm to 100 μm, which may exceed the usual size limits for colloidal particles. An emulsion is termed an oil/water (o/w) emulsion if the dispersed phase is an organic material and the continuous phase is water or an aqueous solution. An emulsion is termed a water/oil (w/o) emulsion if the dispersed phase is water or an aqueous solution and the continuous phase is a water-insoluble liquid such as an “oil.” Emulsions, even very finely divided e.g. micron-scale emulsions, are generally opaque or “milky” in appearance, scattering visible light in a largely wavelength independent manner due to the difference between the refractive index of the dispersed phase and the refractive index of the continuous phase.

Microemulsions are emulsions that are thermodynamically stable systems with a dispersed domain diameter ranging from 1 nm to 250 nm. The use of microemulsions is well known in various arts including pharmacy, petrochemical, agricultural, fragrance, and cosmetics and personal care. Microemulsions provide several advantages over other compositions: they are more stable with regard to density-driven separation, they are less likely to coalescence, and they deliver finely dispersed droplets of lipophilic or amphiphilic active ingredients. As compared to standard emulsions, the dispersed domain droplets of microemulsions are small enough that shorter wavelengths of the visible spectrum are scattered differently than longer wavelengths such that they appear opalescent or hazy and bluish in reflection but nearly clear and orange-tinted in transmission (Rayleigh scattering). In the finest microemulsion dispersions, light in the visible spectrum is transmitted with apparent uniformity and the dispersions appear optically clear to the eye, as apparent solutions, although the presence of discrete domains of immiscible phases may be detected for example by X-ray or electron scattering phenomena.

There is a need in the art to develop improved microemulsions and methods of their manufacture. Particularly, there is a need to develop microemulsions that do not require a co-surfactant, dilutable microemulsions that retain clarity upon dilution, and microemulsions that can be manufactured by methods that do not require high-energy mechanical manipulation such as provided by homogenizers or sonicators. This disclosure satisfies these needs and provides related advantages.

### SUMMARY OF THE INVENTION

The present disclosure provides compositions that include an oil, a surfactant, and optionally a charged polymer complex and methods of manufacturing the compositions. The composition may also include an aqueous phase to provide a microemulsion. The technology further provides methods of use of such compositions in home care and personal care.

In accordance with some embodiments, there are provided charged polymer complex microemulsions comprising: a substantially water-immiscible oil phase; an aqueous phase; a surfactant; and a charged polymer complex. In some embodiments, the charged polymer complex comprises: one or more amphiphilic anionic polymers, one or more cationic charged polymer, or a combination thereof.

In accordance with some embodiments, the microemulsions are optically transparent. In some embodiments, the microemulsions remain optically transparent upon dilution (e.g., dilution with water).

In some embodiments, the microemulsions do not comprise a metal halide salt. In some embodiments, the microemulsions do not comprise a hydrotope.

In accordance with some embodiments, there are provided compositions comprising an oil and a surfactant, wherein the oil has an octanol/water partition coefficient (log Kow) of less than 10; the surfactant HLB is greater than 10; the composition is substantially free of a co-surfactant, a metal halide salt, a hydrotrope, or a combination of two more thereof; the composition is optically transparent; and the composition remains optically transparent upon dilution (e.g., dilution with water).

In some embodiments, the compositions and/or microemulsions do not comprise a co-surfactant.

In some embodiments, the surfactant is selected from the group consisting of an anionic surfactant, a cationic surfactant, an ionic surfactant, a nonionic surfactant, and a zwitterionic surfactant. In some embodiments, the surfactant is selected from the group consisting of: an ethoxylated alcohol represented by the formula R(OC₂H₄)ₙOH, wherein R is a linear, branched, or cyclic alkane moiety; a polyoxyethylene derivative of an ester; a glucoside; and an amphiphilic polymeric material.

In accordance with some embodiments, the surfactant is selected from the group consisting of: cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, sorbitan oleate decylglucoside crosspolymer (SODC), polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hydroxypropyl methylcellulose, carboxymethylcellulose sodium, methylcellulose, hydroxyethylcellulose, hydroxypropylmethyl-cellulose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctyl sodium sulfosuccinate, dialkylesters of sodium sulfosuccinic acid, sodium lauryl sulfate, alkyl aryl polyether sulfonates, mixtures of sucrose stearate and sucrose distearate, C₁₈H₃₇CH₂C(O)N(CH₃)—CH₂(CHOH)₄(CH₂OH)₂, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, random copolymers of vinyl acetate and vinyl pyrrolidone; cationic lipids, ceteth-25, ceteareth-25, PEG-40 hydrogenated castor oil, PPG-5-Ceteth-20, laureth-7, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, ceteareth-20, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅ dimethyl hydroxyethyl ammonium chloride, C₁₂₋₁₅ dimethyl hydroxyethyl ammonium chloride bromide, C₁₂₋₁₃ pareth 9 (P9), coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, Cis trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, polyquaternium-10, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™ ALKAQUAT™, alkyl pyridinium salts; amines, amine salts, amine oxides, imide azolinium salts, protonated quaternary acrylamides, methylated quaternary polymers, and cationic guar.

In some embodiments, the surfactant is selected from the group consisting of: ceteareth-20, ceteth-25, ceteareth-25, PEG-40 hydrogenated castor oil, PPG-5-ceteth-20, laureth-7, PEG-40 stearate, SODC, and P9.

In accordance with some embodiments, the microemulsions further comprise one or more additional surfactants. In some embodiments, the oil phase comprises one or more esters, terpenes, or amides. In some embodiments, the oil phase comprises one or more esters. In some embodiments, the one or more esters are selected from the group consisting of glycerides, fats, and waxes. In some embodiments, the one or more esters are selected from the group consisting of: almond oil, apricot kernel oil, argan oil, avocado oil, babassu oil, baobab oil, black cumin oil, borage oil, broccoli seed oil, beeswax, C₁₂₋₁₅ alkyl benzoate, camelina oil, camellia seed oil, canola oil, capric/caprylic triglycerides (CTG), carrot seed oil, castor oil, chia seed oil, citrus oils, cocoa butter, coconut oil, cranberry seed oil, daikon seed oil, evening primrose oil, flax seed oil, grape seed oil, hazelnut oil, hemp seed oil, jojoba oil, candellila wax, carnauba wax, ozokerite, paraffin, stearin, kokum butter, kukui nut oil, lanolin, macadamia nut oil, mango butter, marula oil, meadowfoam seed oil, medium chain triglycerides, monoi oil, moringa oil, neem oil, octyl methoxycinnamate, octocrylene, olive oil, palm fruit oil, palm kernel oil, pomegranate seed oil, prickly pear seed oil, pumpkin seed oil, red palm oil, raspberry seed oil, rice bran oil, rosehip oil, sacha inchi oil, safflower oil, seabuckthorn fruit oil, sesame seed oil, shea nut oil, shorea butter, soybean oil, strawberry seed oil, sunflower oil, tamanu oil, walnut oil, wheat germ oil, and chemically modified, esterified, partially de-esterified or hydrogenated forms thereof. In some embodiments, the one or more esters are selected from the group consisting of: argan oil, CTG, citrus oil, octyl methoxycinnamate, and octocrylene.

In some embodiments, the oil comprises a-pinene, camphene, b-pinene, sabinene, myrcene, a-terpinene, linalool, b-bisabolene, limonene, trans-a-bergamotene, nerol neral, or a combination of two or more thereof.

In some embodiments, the composition is a triglyceride solubilizer.

In some embodiments, the compositions further include an aqueous phase and the compositions are microemulsions.

In accordance with some embodiments, the microemulsions have a dispersed phase domain diameter of 250 nm or less. In some embodiments, the microemulsions have a dispersed phase domain diameter of 100 nm or less. In some embodiments, the microemulsions have a dispersed phase domain diameter of 75 nm or less. In some embodiments, the microemulsions have a dispersed phase domain diameter of 50 nm or less.

In accordance with some embodiments, when the aqueous phase is present the concentration of the oil phase is about 0.01% to about 50% by weight of the microemulsion. In some embodiments, the concentration of the oil phase is about 0.1% to about 10% by weight of the microemulsion. In some embodiments, the concentration of the aqueous phase is about 30% to about 95% by weight of the microemulsion. In some embodiments, the concentration of the aqueous phase is about 50% to about 80% by weight of the microemulsion. In some embodiments, the concentration of the surfactant is about 0.5% to about 80% by weight of the microemulsion. In some embodiments, the concentration of the surfactant is about 15% to about 40% by weight of the microemulsion. In some embodiments, the concentration of the amphiphilic anionic charged polymer is about 0.1% to about 50% by weight of the microemulsion. In some embodiments, the concentration of the amphiphilic anionic charged polymer is about 10% to about 30% by weight of the microemulsion. In some embodiments, the concentration of the amphiphilic cationic charged species is about 0.1% to about 50% by weight of the microemulsion. In some embodiments, the concentration of the amphiphilic cationic charged species is about 10% to about 30% by weight of the microemulsion. In some embodiments, the microemulsion further comprises at least one amphiphilic charged species selected from the group consisting of: proteins, products of protein hydrolysis, peptides, amino acids, nucleic acids, oligomers, plasmids, sense- and anti-sense ribonucleic acid and deoxyribonucleic acid sequences, and conjugates of proteins and nucleic acids.

In some embodiments, the one or more amphiphilic anionic charged polymers are selected from the group consisting of: acacia gum, agar, polyacrylic acid, albumin, alginic acid, carbomer, carboxymethylcellulose, carrageenan, cassia gum, cellulose gum, chondroitin, curdlan, gelatin, dextran, fibrin, fulcelleran, gellan gum, ghatti gum, gum tragacanth, heparin, hyaluronic acid, karaya gum, locust bean gum, pea protein, pectin, polyoxyethylene-polyoxypropylene, synthetic block copolymers, pullulan, saponins, starch, tara gum, whey protein, xanthan gum, zein, ions and salts thereof, polymeric materials of molecular weight above 1000 amu having at least two carboxylic acid reactive groups, and combinations thereof. In some embodiments, the one or more amphiphilic anionic charged polymers are selected from the group consisting of: alginic acid, carrageenan, and hyaluronic acid ions and salts thereof.

In accordance with some embodiments, the amphiphilic cationic charged polymer is selected from the group consisting of benzalkonium, cetylpyridinium, chitosan, cocodimonium hydroxypropyl hydrolyzed keratin, hydroxypropyltrimonium hydrolyzed wheat protein, hydroxypropyl oxidized starch PG-trimonium, PEG-3 dioleylamidoethylmonium, laurdimoniumhydroxypropyl decylglucosides, polyquaternium-10, polyquaternium-11, polyquaternium-78, polyquaternium-80, polyquaternium-81, polyquaternium-88, polyquaternium-101, quaternium-79 hydrolyzed silk protein, silicone quaternium-17, silicone quaternium-8, starch hydoxypropyltrimonium, steardimonium hydroxyethylcellulose, steardimonium hydroxypropyl panthenyl PEG-7 dimethicone, cocodimonium hydroxyethylcellulose, polyvinylamine, water-soluble quaternary amines, and amphiphilic Lewis bases. In some embodiments, the one or more amphiphilic cationic charged polymers are selected from the group consisting of: chitosan, polyquaternium-10, hydroxypropyl oxidized starch PG-trimonium, and cationic guar.

In some embodiments, the charged polymer complex encapsulates the dispersion phase. In some embodiments, the charged polymer complex forms an interconnected network of fiber-like structures.

In some embodiments, the compositions and/or microemulsions further comprise one or more dyes. In some embodiments, the one or more dyes are selected from the group consisting of: a temporary non-oxidative hair dye, a semi-permanent non-oxidative hair dye, and a permanent oxidative hair dye. Nonlimiting examples of temporary non-oxidative hair dye include but are not limited to pyrazol (acid yellow 23), monoazo (acid orange 7), nitro (acid yellow 1), monoazo (acid red 33), xanthene (acid red 92), anthraquinone (acid violet 43), triphenylmethane (acid blue 9), diazo (acid black 1). Nonlimiting examples of a semi-permanent non-oxidative hair dye include but are not limited to chemical classification: nitro-aniline, HC yellow no. 2, HC red no. 3, 4-hydroxypropylamino-3-nitrophenol, NN-bis-(2 hydroxyethyl)-2-nitro phenylenediamine, hc blue no. 2, the cationic dyes (direct dye), basic red 51, basic red 76, basic brown 16, basic brown 17, basic blue 99, and basic yellow 57. Nonlimiting examples of a permanent oxidative hair dye include but are not limited to couplers, 4-chlororesorcinol, 2,4-diaminophenoxyethanol hcl, 2-amino-hydroxyethylaminoanisole sulfate, 4-amino-2 hydroxytoluene, m-aminophenol, and resorcinol.

In accordance with some embodiments, the compositions and/or microemulsions further comprise one or more photo-absorbers. In some embodiments, the one or more photo-absorbers are present in the oil phase. In some embodiments, the photo-absorber comprises one or more materials selected from the group consisting of p-aminobenzoic acid, avobenzone, 3-benzylidine camphor, bismidazylate, diethylamino hydroxybenzoyl hexyl benzoate, diethylhexyl butamido triazone, dimethicodiethylbenzal malonate, ecamsule, ensulizole, homosalate, isoamyl p-methoxycinnamate, 4-methylbenzylidine camphor, octocrylene, octyl dimethyl p-aminobenzoic acid, octyl methoxycinnamate, octyl salicylate, octyl triazone, bis-ethylhexyloxyphenol methoxyphenyl triazine, methylene bis-benzotriazolyl tetramethylbutylphenol, oxybenzone, polyacrylamidomethyl benzylidine camphor, and sulisobenzone.

In some embodiments, the compositions and/or microemulsions further comprise one or more vitamins or nutritional supplements. In some embodiments, the one or more vitamins or nutritional supplements are selected from the group consisting of: 2-methyl-1,4-naphthoquinone (3-) derivatives, 22-dihydroergocalciferol, and cyanocobolamins, and omega-3-fatty acids, ascorbic acid, biotin, carotinoids, cholocalciferol, curcumin, ergocalciferol, ergosterol, esterified ascorbates, fat-soluble forms of thiamin, folates, iron, lumisterol, niacin, pantothenic acid, pyridoxine, retinol, riboflavin, secosteroids, sitocalciferol, tocopherol, and ubiquinone.

In accordance with some embodiments, the compositions and/or microemulsions further comprise one or more insect repellents. In some embodiments, the one or more insect repellents are selected from the group consisting of: ethyl butylacetylaminopropionate, benzaldehyde, N,N-diethyl-m-toluamide (DEET), dimethyl carbate, dimethyl phthalate, hydroxyethyl isobutyl piperidine carboxylate (Icaridin), indalone, metofluthrin, permethrin, tricyclodecenyl allyl ether, birch (Betula sp) bark, bog myrtle (Myrica gale), catnip extracts, citronella oil, citrus oils, limonene, lemon eucalyptus (Corymbia citriodora) oil, neem oil, lemongrass oil, and tea tree oil.

In some embodiments, the citrus oils include, but are not limited to, lemon peel oil, orange peel oil, lime oil peel, grapefruit oil peel, and combinations of two or more thereof. In some embodiments, the citrus oils include lemon peel oil.

In some embodiments, the compositions and/or microemulsions further comprise one or more gelling agents. In some embodiments, the one or more gelling agents comprise at least one fatty acid with a melting point above 35° C. In some embodiments, the one or more gelling agents comprise at least one polyamide.

In some embodiments, the compositions and/or microemulsions further comprise an uncharged, water-soluble additive. In some embodiments, the uncharged water-soluble additive is selected from the group consisting of: antioxidants, bleaches, cosmetic materials, fragrances, humectants, and pharmacologic agents.

In accordance with some embodiments, there are provided personal care compositions comprising the compositions and/or microemulsions. In some embodiments, the personal care composition is a hair care composition. In some embodiments, the hair care composition is selected from the group consisting of: shampoo, conditioner, treatment, mask, styling agent or color protecting treatments and technologies. In some embodiments, the hair care composition softens the hair including head and body hair (e.g., beard hair). In some embodiments, the composition is a skin care composition. In some embodiments, the composition is a moisturizer, cream, lotion, or body oil. In some embodiments, the oil phase comprises one or more of the group consisting of: coconut oil, argan oil, CTG, citrus oil, and octyl methoxycinnamate. In some embodiments, the surfactant is P9 or SODC.

In some embodiments, the composition further comprises at least one peptide. In some embodiments, the peptide is a pentapeptide comprising amino acids selected from the group consisting of: cysteine, arginine, proline, and serine. In some embodiments, the pentapeptide has an amino acid sequence consisting of CCRPS (SEQ ID NO: 1).

In accordance with some embodiments, there are provided methods of treating a surface, the methods comprising applying the compositions and/or microemulsions, thereby treating the surface. In some embodiments, the surface comprises a textile. In some embodiments, the surface comprises a kitchen surface. In some embodiments, the kitchen surface comprises at least one of the group consisting of: floor, countertop, stovetop, sink, and appliance. In some embodiments, the surface comprises leather, vinyl, a synthetic leather material. In some embodiments, the surface comprises a wall, floor, or ceiling. In some embodiments, the surface comprises a plant.

In accordance with some embodiments, there are provided methods of treating hair, the methods comprising applying the compositions and/or microemulsions, thereby treating the hair. In some embodiments, the hair is artificially colored. In some embodiments, the treatment prevents or reduces washout of the artificial color. In some embodiments, the treatment comprises repair or prevention of hair damage. In some embodiments, the hair damage comprises split-ends. In some embodiments, the personal care composition is applied to the hair for about 30 seconds to about 5 minutes. In some embodiments, the method further comprises washing the personal care composition out of the hair following application. In some embodiments, the treatment comprises deposition of a photo-absorber.

In accordance with some embodiments, there are provided methods of treating an internal or external surface of a subject, the methods comprising applying the personal care composition comprising the compositions and/or microemulsions, thereby treating the internal or external surface. In some embodiments, the internal surface is one or more of the group selected from: teeth, oral cavity, and mucosal surface. In some embodiments, the external surface is one or more of the group selected from: skin, nail, and scalp. In some embodiments, the external surface is a nail comprising a fingernail or a toenail. In some embodiments, the external surface is skin. In some embodiments, the external surface is hair. In some embodiments, the treatment comprises deposition of a photo-absorber.

In accordance with some embodiments, there are provided methods of laundry care, the methods comprising applying the compositions and/or microemulsions to laundry. In some embodiments, the method further comprises removal of one or more stains from the laundry. In some embodiments, the removal comprises spot cleaning. In some embodiments, the method further comprises prevention of soil-redeposition.

In accordance with some embodiments, there are provided methods of preparing an encapsulated microemulsion, the methods comprising admixing: a substantially water-immiscible oil phase; an aqueous phase; a surfactant; one or more amphiphilic anionic charged polymers; and one or more amphiphilic cationic charged polymers. In some embodiments, the admixing of (a)-(e) is simultaneous, sequential, or consecutive. In some embodiments, the admixing of (a)-(e) is simultaneous, sequential, or consecutive.

In accordance with some embodiments, the oil phase (a) is admixed with the surfactant phase (c), and the to the resulting mixture is added in sequence, the aqueous phase (b), anionic phase (d), and cationic phase (e). In some embodiments, the anionic phase (d) is added after the cationic phase (e). In some embodiments, the aqueous phase (b) is admixed with the surfactant phase (c), and the to the resulting mixture is added in sequence, the oil phase (d), anionic phase (d), and cationic phase (e). In some embodiments, the aqueous phase (b) is admixed with the surfactant phase (c), and the to the resulting mixture is added in sequence, anionic phase (d), cationic phase (e), and the oil phase (d). In some embodiments, the oil, aqueous, and surfactant phases, (a), (b), and (c) are admixed simultaneously, followed by the addition of the anionic phase (d), and then the cationic phase (e). In some embodiments, the oil, aqueous, and surfactant phases, (a), (b), and (c) are admixed simultaneously, followed by the addition of the cationic phase (e), and then the anionic phase (d).

In accordance with some embodiments, the surfactant and the oil are admixed to provide a mixture. In some embodiments, the admixing is simultaneous, sequential, or consecutive. In some embodiments, an aqueous phase is added to the mixture.

In accordance with some embodiments, the compositions and/or microemulsions are dilutable (i.e., dilutable compositions and/or microemulsions), wherein the composition and/or microemulsion is optically transparent, the composition and/or microemulsion remains transparent upon dilution, and the composition and/or microemulsion does not contain co-surfactants, metal halides, or hydrotopes.

In some embodiments, the dilutable composition and/or microemulsion further comprise at least one polymer. In some embodiments, the at least one polymer is ionically charged.

In some embodiments, the charged polymer is anionic. In some embodiments, the anionic polymer is selected from the group consisting of the following Lewis acids and their anions and water-soluble salts: alginic acid, arabic acid, carboxymethylcellulose, carrageenan, saponins, carbomers and related polymers and block copolymers bearing carboxylic acid moieties, collagen, hyaluronic acid, gellan gum, pectin, and generally, polymeric materials of molecular weight above 1000 amu presenting at least two carboxylic acid reactive groups, or combinations of these materials.

In some embodiments, the charged polymer is cationic. In some embodiments, the cationic polymer is selected from the group consisting of the following Lewis bases, anions, and their water-soluble salts: benzalkonium, cetylpyridinium, chitosan, cocodimonium hydroxypropyl hydrolyzed keratin, cocoglucosides hydroxypropyl, hydroxypropyltrimonium hydrolyzed wheat protein, hydroxypropyl oxidized starch PG-trimonium, PEG-3 dioleylamidoethylmonium, laurdimoniumhydroxypropyl decylglucosides, polyquaternium-10, polyquaternium-11, polyquaternium-78, polyquaternium-80, polyquaternium-81, polyquaternium-88, polyquaternium-101, quaternium-79 hydrolyzed silk protein, silicone quaternium-17, silicone quaternium-8, starch hydoxypropyltrimonium, steardimonium hydroxyethylcellulose, steardimonium hydroxypropyl panthenyl PEG-7 dimethicone, cocodimonium hydroxyethylcellulose, polyvinylamine and generally, any water-soluble quaternary amine or similar Lewis base, or combinations of any of these materials. The dilutable microemulsion claim 1 or 2, wherein at least one polymer is not ionically charged. The dilutable microemulsion of claim 5, wherein the uncharged polymer is selected from the group consisting of agar, acrylic acid, albumins, carrageenans, casein, cellulose gums, including hydroxypropylmethyl cellulose, hydroxypropyl cellulose, and hydroxyethyl cellulose (HEC), methylcellulose and microcrystalline cellulose, chitin derivatives, chondroitin, curdlan, gelatin, dextran, fibrin, fulcelleran, gellan gum, ghatti gum, guar gum, gum tragacanth, heparin, hyaluronic acid, karaya gum, locust bean gum, pea protein, pectin, polyoxyethylene-polyoxypropylene and other synthetic block copolymers, pullulan, starch, soy protein, whey protein, xanthan gum, and zein, polyethylene glycols, polypropylene glycols, poloxamers, poloxamines, polybutylene glycols, polyvinylpyrrolidones, polyvinyl alcohols, polyacrylic acids, polymers of biological organic acids, including poly-lactic acid, poly-lactic co-glycolic acid, starches and starch derivatives, including hydroxypropyl starch, proteins, including partially hydrolyzed proteins, and polypeptides, and combinations and derivatives thereof.

In some embodiments, the composition and/or microemulsion having an aqueous phase has a dispersed phase domain diameter of 250 nm or less. In some embodiments, the composition and/or microemulsion having an aqueous phase has a dispersed phase domain diameter of 200 nm or less. In some embodiments, the composition and/or microemulsion having an aqueous phase has a dispersed phase domain diameter of 150 nm or less. In some embodiments, the composition and/or microemulsion having an aqueous phase has a dispersed phase domain diameter of 100 nm or less. In some embodiments, the composition and/or microemulsion having an aqueous phase has a dispersed phase domain diameter of 75 nm or less. In some embodiments, the composition and/or microemulsion having an aqueous phase has a dispersed phase domain diameter of 50 nm or less.

In some embodiments, the surfactant is selected from the group consisting of an anionic surfactant, a cationic surfactant, an ionic surfactant, a nonionic surfactant, and a zwitterionic surfactant. In some embodiments, the surfactant is selected from the group consisting of: (a) an ethoxylated alcohol represented by the formula R(OC₂H₄)ₙOH, wherein R is a linear, branched, or cyclic alkane moiety, (b) a polyoxyethylene derivative of an ester, (c) a glucoside, and (d) an amphiphilic polymeric material.

In some embodiments, the surfactant is selected from the group consisting of: cetyl pyridinium chloride, phosphatides, dextran, cholesterol, stearic acid, benzalkonium chloride, glycerol monostearate, cetostearyl alcohol, cetomacrogol, emulsifying wax, sorbitan esters, polyoxyethylene alkyl ethers, polyoxyethylene castor oil derivatives, polyoxyethylene sorbitan fatty acid esters, sorbitan oleate decylglucoside crosspolymer (SODC), polyethylene glycols, dodecyl trimethyl ammonium bromide, polyoxyethylene stearates, phosphates, sodium dodecylsulfate, triethanolamine, poloxamers, poloxamines, charged phospholipids, sulfosuccinates, sodium lauryl sulfate, alkyl aryl polyether sulfonates, mixtures of sucrose stearate and sucrose distearate, decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside, PEG-phospholipid, PEG-cholesterol, PEG-cholesterol derivative, PEG-vitamin A, PEG-vitamin E, copolymers of vinyl acetate and vinyl pyrrolidone; cationic lipids, ceteth-25, ceteareth-25, PEG-40 hydrogenated castor oil, PPG-5-Ceteth-20, laureth-7, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, ceteareth-20, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅ dimethyl hydroxyethyl ammonium chloride, C₁₂₋₁₅ dimethyl hydroxyethyl ammonium chloride bromide, C₁₂₋₁₃ pareth 9 (P9), coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄-18)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, Cis trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, polyquaternium-10, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™ ALKAQUAT™, alkyl pyridinium salts, betaines, amines, amine salts, and amine oxides.

In some embodiments, the surfactant is selected from the group consisting of: ceteareth-20, ceteth-25, ceteareth-25, PEG-40 hydrogenated castor oil, PPG-5-ceteth-20, laureth-7, PEG-40 stearate, SODC, and P9.

In some embodiments, the composition and/or microemulsion having an aqueous phase further comprise one or more additional surfactants.

In some embodiments, the oil/oil phase comprises one or more alkanes, silicones, organosilicones, esters, terpenes, or amides, or halogen-substituted derivatives thereof. In some embodiments, the oil phase comprises one or more esters. In some embodiments, the one or more esters are selected from the group consisting of glycerides, fats, oils, and waxes. In some embodiments, the oil comprises a-pinene, camphene, b-pinene, sabinene, myrcene, a-terpinene, linalool, b-bisabolene, limonene, trans-a-bergamotene, nerol neral, or a combination of two or more thereof.

In some embodiments, the one or more esters, fats, oils, and waxes are selected from the group consisting of: almond oil, apricot kernel oil, argan oil, avocado oil, babassu oil, baobab oil, black cumin oil, borage oil, broccoli seed oil, beeswax, C₁₂₋₁₅ alkyl benzoate, buruti oil, camelina oil, camellia seed oil, canola oil, capric/caprylic triglycerides (CTG), carrot seed oil, castor oil, chia seed oil, citrus oils, cocoa butter, coconut oil, cranberry seed oil, daikon seed oil, evening primrose oil, flax seed oil, grape seed oil, hazelnut oil, hemp seed oil, jojoba oil, kokum butter, kukui nut oil, lanolin, macadamia nut oil, mango butter, marula oil, meadowfoam seed oil, medium chain triglycerides (MCT), monoi oil, moringa oil, neem oil, octyl methoxycinnamate, octocrylene, olive oil, palm fruit oil, palm kernel oil, pomegranate seed oil, prickly pear seed oil, pumpkin seed oil, red palm oil, raspberry seed oil, rice bran oil, rosehip oil, sacha inchi oil, safflower oil, seabuckthorn fruit oil, sesame seed oil, shea nut oil, shorea butter, soybean oil, strawberry seed oil, sunflower oil, tamanu oil, walnut oil, wheat germ oil, and chemically modified, esterified, partially de-esterified or hydrogenated forms thereof, and esters of organic acids, including acetic, propionic, butyric, valeric, caproic, lactic, malic, citric and benzoic acid esters, and simple esters of fatty acids.

In some embodiments, the one or more esters are selected from the group consisting of: argan oil, coconut oil, shea nut oil, mango butter, sunflower oil, flax seed oil, CTG, citrus oil, and MCT.

The dilutable microemulsion, wherein the one or more amphiphilic anionic charged polymers are selected from the group consisting of: acacia gum, agar, alginate, polyacrylic acid, albumin, carbomer, carrageenan, cassia gum, cellulose gum, chondroitin, curdlan, gelatin, dextran, fibrin, fulcelleran, gellan gum, ghatti gum, gum tragacanth, heparin, hyaluronic acid, karaya gum, locust bean gum, pea protein, pectin, polyoxyethylene-polyoxypropylene, synthetic block copolymers, pullulan, saponins, starch, tara gum, whey protein, soy protein, rice protein, silk protein, and hydrolysates of these proteins, xanthan gum, zein, ions and salts thereof, polymeric materials of molecular weight above 1000 amu having at least two carboxylic acid reactive groups, and combinations thereof.

In some embodiments, the one or more amphiphilic anionic charged polymers are selected from the group consisting of: alginate, cellulose gum, hyaluronic acid, and carrageenan.

In some embodiments, the amphiphilic cationic charged polymer is selected from the group consisting of benzalkonium, cetylpyridinium, chitosan, cocodimonium hydroxypropyl hydrolyzed keratin, cocoglucosides hydroxypropyl, hydroxypropyl guar trimonium chloride, hydroxypropyltrimonium hydrolyzed wheat protein, hydroxypropyl oxidized starch PG-trimonium, PEG-3 dioleylamidoethylmonium, laurdimoniumhydroxypropyl decylglucosides, polyquaternium-10, polyquaternium-11, polyquaternium-78, polyquaternium-80, polyquaternium-81, polyquaternium-88, polyquaternium-101, quaternium-79 hydrolyzed silk protein, silicone quaternium-17, silicone quaternium-8, starch hydoxypropyltrimonium, steardimonium hydroxyethylcellulose, steardimonium hydroxypropyl panthenyl PEG-7 dimethicone, cocodimonium hydroxyethylcellulose, polyvinylamine, water-soluble quaternary amines, and amphiphilic Lewis bases.

In some embodiments, the one or more amphiphilic cationic charged polymers are selected from the group consisting of: chitosan, cocodimonium hydroxyethylcellulose, hydroxypropyl guar trimonium chloride, and polyquaternium-10.

In some embodiments, when an aqueous phase is present the concentration of the oil phase is about 0.01% to about 50% by weight of the dilutable microemulsion and/or composition. In some embodiments, the concentration of the oil phase is about 0.1% to about 10% by weight of the dilutable microemulsion. In some embodiments, the concentration of the aqueous phase is about 30% to about 98% by weight of the dilutable microemulsion and/or composition. In some embodiments, the concentration of the aqueous phase is about 50% to about 90% by weight of the dilutable microemulsion and/or composition. In some embodiments, the concentration of the surfactant is about 0.1% to about 80% by weight of the dilutable microemulsion and/or composition. In some embodiments, the concentration of the surfactant is about 1% to about 40% by weight of the dilutable microemulsion and/or composition. In some embodiments, the concentration of the amphiphilic anionic charged polymer is about 0.01% to about 10% by weight of the dilutable microemulsion and/or composition. In some embodiments, the concentration of the amphiphilic anionic charged polymer is about 0.1% to about 1% by weight of the dilutable microemulsion and/or composition. In some embodiments, the concentration of the amphiphilic cationic charged species is about 0.01% to about 10% by weight of the dilutable microemulsion and/or composition. In some embodiments, the concentration of the amphiphilic cationic charged species is about 0.1% to about 1% by weight of the dilutable microemulsion and/or composition.

In some embodiments, the dilutable microemulsion and/or composition further comprises at least one amphiphilic charged species selected from the group consisting of: proteins, products of protein hydrolysis, peptides, amino acids, nucleic acids, oligomers, plasmids, sense- and anti-sense ribonucleic acid and deoxyribonucleic acid sequences, and conjugates of proteins and nucleic acids.

In some embodiments, the dilutable microemulsion and/or composition further comprise one or more photo-absorbers. In some embodiments, the one or more photo-absorbers are present in the oil phase. In some embodiments, the one or more photo-absorbers are present in the aqueous phase. In some embodiments, the one or more photo-absorbers are substantially present in both the oil and the aqueous phases. In some embodiments, the photo-absorber comprises one or more materials selected from the group consisting of p-aminobenzoic acid, avobenzone, 3-benzylidine camphor, bismidazylate, diethylamino hydroxybenzoyl hexyl benzoate, diethylhexyl butamido triazone, dimethicodiethylbenzal malonate, ecamsule, ensulizole, homosalate, isoamyl p-methoxycinnamate, 4-methylbenzylidine camphor, octocrylene, octyl dimethyl p-aminobenzoic acid, octyl methoxycinnamate, octyl salicylate, octyl triazone, bis-ethylhexyloxyphenol methoxyphenyl triazine, methylene bis-benzotriazolyl tetramethylbutylphenol, oxybenzone, polyacrylamidomethyl benzylidine camphor, and sulisobenzone.

In some embodiments, the dilutable microemulsion and/or composition further comprise one or more vitamins or nutritional supplements. In some embodiments, the one or more vitamins or nutritional supplements are selected from the group consisting of: 2-methyl-1,4-naphthoquinone (3-) derivatives, 22-dihydroergocalciferol, and cyanocobolamins, and omega-3-fatty acids, ascorbic acid, biotin, carotinoids, cholocalciferol, curcumin, ergocalciferol, ergosterol, esterified ascorbates, fat-soluble forms of thiamin, folates, iron, lumisterol, niacin, pantothenic acid, pyridoxine, retinol, riboflavin, secosteroids, sitocalciferol, tocopherol, and ubiquinone.

In some embodiments, the dilutable microemulsion and/or composition further comprise one or more insect repellents. In some embodiments, the one or more insect repellents are selected from the group consisting of: ethyl butylacetylaminopropionate, benzaldehyde, N,N-diethyl-m-toluamide (DEET), dimethyl carbate, dimethyl phthalate, hydroxyethyl isobutyl piperidine carboxylate (Icaridin), indalone, metofluthrin, permethrin, tricyclodecenyl allyl ether, birch (Betula sp) bark, bog myrtle (Myrica gale), catnip extracts, citronella oil, citrus oils, limonene, lemon eucalyptus (Corymbia citriodora) oil, neem oil, lemongrass oil, and tea tree oil.

In some embodiments, the dilutable microemulsion and/or composition further comprise one or more gelling agents. In some embodiments, the one or more gelling agents comprise at least one fatty acid with a melting point above 35° C. In some embodiments, the one or more gelling agents comprise at least one polyamide.

In some embodiments, the dilutable microemulsion and/or composition further comprise an uncharged, water-soluble additive. In some embodiments, the uncharged water-soluble additive is selected from the group consisting of: antioxidants, bleaches, cosmetic materials, fragrances, humectants, and pharmacologic agents.

In some embodiments, the dilutable microemulsion and/or composition is substantially free of a charged polymer complex. In some embodiments, the dilutable microemulsion and/or composition does not contain a charged polymer complex.

In some embodiments, there are provided personal care compositions comprising a dilutable microemulsion and/or composition as described herein.

In some embodiments of the personal care composition, the composition is a hair care composition. In some embodiments of the personal care composition, the hair care composition is selected from the group consisting of: shampoo, conditioner, treatment, mask, styling agent, or color protecting treatments and technologies.

In some embodiments of the personal care composition, the composition is a skin care composition. In some embodiments of the personal care composition, the composition is a moisturizer, cream, lotion, or body oil.

In some embodiments of the personal care composition, the oil/oil phase comprises one or more of the group consisting of: coconut oil, citrus oil, CTG, and octyl methoxycinnamate

In some embodiments of the personal care composition, the surfactant is P9 or SODC.

In some embodiments of the personal care composition, the composition further comprises at least one peptide.

In some embodiments, there are provided methods of treating a surface, the method comprising applying the composition and/or microemulsion as described herein to the surface, thereby treating the surface. In some embodiments, the surface comprises a textile. In some embodiments, the surface comprises a kitchen surface. In some embodiments, the kitchen surface comprises at least one of the group consisting of: floor, countertop, stovetop, sink, and appliance. In some embodiments, the surface comprises leather, vinyl, a synthetic leather material. In some embodiments, the surface comprises a wall, floor, or ceiling. In some embodiments, the surface comprises a plant.

In some embodiments, there are provided methods of treating hair, the method comprising applying a personal care composition according to any of the embodiments described herein to the hair, thereby treating the hair. In some embodiments, the treatment comprises repair or prevention of hair damage. In some embodiments, the personal care composition is applied to the hair for about 30 seconds to about 5 minutes.

In some embodiments, the methods further comprise washing the personal care composition out of the hair following application.

In some embodiments, there are provided methods of treating an internal or external surface of a subject, the method comprising applying a personal care composition as described herein to the internal or external surface of the subject, thereby treating the internal or external surface. In some embodiments, the internal surface is one or more of the group selected from: teeth, oral cavity, and mucosal surface. In some embodiments, the external surface is one or more of the group selected from: skin, nail, and scalp. In some embodiments, the external surface is a nail comprising a fingernail or a toenail. In some embodiments, the external surface is skin. In some embodiments, the external surface is hair. In some embodiments, the treatment comprises deposition of a photo-absorber.

In some embodiments, there are provided methods of laundry care, the method comprising applying any one of the composition and/or microemulsion described herein to laundry. In some embodiments, the methods further comprise removal of one or more stains from the laundry. In some embodiments, the methods further comprise prevention of soil-redeposition.

In some embodiments, there are provided methods of preparing a dilutable microemulsion, the method comprising admixing: (a) a substantially water-immiscible oil phase; (b) an aqueous phase; (c) a surfactant; (d) one or more amphiphilic charged polymers; (e) and/or one or more uncharged polymers. In some embodiments, the admixing of (a)-(e) is simultaneous, sequential, or consecutive. In some embodiments, the oil phase (a) is admixed with the surfactant phase (c), and the to the resulting mixture is added in sequence, the aqueous phase (b), charged polymer phase (d), and uncharged phase (e). In some embodiments, the oil phase (a) is admixed with the surfactant phase (c), and the to the resulting mixture is added in sequence, the aqueous phase (b), uncharged polymer phase (e), and charged polymer phase (d). In some embodiments, the aqueous phase (b) is admixed with the surfactant phase (c), and the to the resulting mixture is added in sequence, the oil phase (d), charged polymer phase (d), and uncharged polymer phase (e). In some embodiments, the aqueous phase (b) is admixed with the surfactant phase (c), and the to the resulting mixture is added in sequence, charged polymer phase (d), uncharged polymer phase (e), and the oil phase (d). In some embodiments, the oil, aqueous, and surfactant phases, (a), (b), and (c) are admixed simultaneously, followed by the addition of the charged polymer phase (d), and then the uncharged polymer phase (e). In some embodiments, the oil, aqueous, and surfactant phases, (a), (b), and (c) are admixed simultaneously, followed by the addition of the uncharged polymer phase (e), and then the charged polymer phase (d).

### BRIEF DESCRIPTION OF THE DRAWINGS

FIG. 1. This image depicts a transmission electron micrograph of a microencapsulated microemulsion prepared according to the disclosed methods, and dried onto a standard TEM sample grid. The scale bar represents 400 nm. A lattice-like network of interconnecting strands is apparent. The network of strands are believed without limitation to be the result of association of the anionic and cationic polymers described herein.

FIG. 2. This image depicts a transmission electron micrograph of a microemulsion prepared without encapsulating polymers, according to the disclosed methods, and dried onto a standard TEM sample grid. The scale bar represents 400 nm. In this representative image, the absence of any lattice-like network of interconnecting strands may be observed.

FIG. 3. This image depicts a transmission electron micrograph (TEM) of a microencapsulated microemulsion prepared according to the disclosed methods, and dried onto a standard TEM sample grid. The scale bar represents 3 μm. In this image, some larger droplets are observed, and it is informative to see a cloudlike assemblage of what are understood without limitation to be the associated cationic and anionic polymers that form the encapsulating structures of the composition. The structure of this encapsulating layer is believed to be more evident in the larger particles due to their size relative to the resolution limit of the microscope. At the lower center of the image, a diffuse association between two particles can be seen, that is understood without limitation to be similar to the associations that form the network visible at higher magnification.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

### Definitions

As used herein, the singular forms “a,” “an,” and “the” designate both the singular and the plural, unless expressly stated to designate the singular only.

It is to be understood, although not always explicitly stated, that all numerical designations are preceded by the term “about.” The term “about” means that the number comprehended is not limited to the exact number set forth herein, and is intended to refer to numbers substantially around the recited number while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 15%, 10%, 5%, 1%, 0.1% or 0.01% of the particular term.

Also as used herein, “and/or” refers to and encompasses any and all possible combinations of one or more of the associated listed items, as well as the lack of combinations when interpreted in the alternative (“or”).

The terms “apply,” “application,” or “applying” as used herein refer to providing, giving, administering, or contacting a target with a composition of the present disclosure. Administration shall include without limitation, administration by oral, inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of application.

As used herein, the term “charged polymer complex” refers to a structure formed by an adduct of a Lewis base and a complementary Lewis acid. In some embodiments, the charged polymer complex comprises a wall and/or cap that encapsulates the dispersion phase of a microemulsion. In some embodiments, the charged polymer complex comprises interconnected fibers. In some embodiments, the charged polymer complex comprises both a wall and/or cap and interconnected fibers. In some embodiments, the charged polymer complex is insoluble.

As used herein, the term “Lewis acid” a molecular entity and the corresponding chemical species that is an electron-pair acceptor and therefore able to react with a Lewis base to form a Lewis adduct, by sharing the electron pair furnished by the Lewis base. In some embodiments, a Lewis acid is an amphiphilic cationic species. As used herein, a “Lewis base” is a molecular entity (and the corresponding chemical species) able to provide a pair of electrons and thus capable of coordination to a Lewis acid, thereby producing a Lewis adduct. In some embodiments, a Lewis base is an amphiphilic anionic polymer. Non-limiting, exemplary Lewis acids and Lewis bases suitable for use in the disclosed microemulsions are provided herein. Unless otherwise specified, or clear from the context, mention of the presence of a particular Lewis acid or a particular Lewis base as a component of a composition will be understood to encompass either the free acid or base, or the salts of these.

As used herein, the term “microemulsion” refers to an emulsion that is thermodynamically stable and with a dispersed domain diameter ranging from 1 nm to 250 nm. The microemulsions of the present disclosure may be o/w or w/o and are not limited to a particular dispersion phase structure. For example, microemulsions of the present disclosure may comprise droplets, microdroplets, and/or bicontinuous microstructure. In some embodiments, the microemulsions of the present disclosure are suitable for pharmaceutical, petrochemical, agricultural, home care, fragrance, cosmetic, and personal care use.

As used herein, the term “surfactant” refers to a substance which lowers the surface tension of the medium in which it is dissolved, and/or the interfacial tension with other phases. Accordingly, the surfactant is positively adsorbed at the liquid/vapor or at other interfaces. Non-limiting, exemplary surfactants suitable for use in the disclosed compositions and/or microemulsions are provided herein.

In the context of an animal subject (e.g., a human), the terms “treat”, “treating” or “treatment” refer to alleviating, abating, or ameliorating one or more conditions, prophylactically preventing the development of one or more conditions, or imparting a desired property to the treated surface of the subject. Non-limiting examples of conditions include one or more of the following: hair damage, split ends, dry hair, dry skin, pruritis, eczema, aging, sun damage, chemical damage, loss of artificial hair color, loss of moisture, oil build up, vitamin deficiency, and enamel stains. Non-limiting examples of a desired property include adding moisture (e.g., via occlusive action, humefactant action, or through restoration of deficient materials), shine, enamel whiteness, protection from ultraviolet light, absorbing or reflecting ultraviolet light, resistance to accumulation of dirt, resistance to damage, repelling insects, and wound healing. In the context of an inanimate target surface (e.g., a kitchen surface), the terms “treat”, “treating” or “treatment” refer to alleviating, abating, or ameliorating one or more conditions, prophylactically preventing the development of one or more conditions, or imparting a desired property to the treated surface. Non-limiting examples of conditions include one or more of the following: damage, scratches, dryness, soil build-up, fading, and loss of color. Non-limiting examples of a desired property include adding moisture, shine, cleanliness, lubrication, brightness, protection from ultraviolet light, absorbing or reflecting ultraviolet light, resistance to accumulation of soil, strength, and resistance to damage.

As used herein, the term “subject” is used interchangeably with “patient,” and indicates an animal such as a mammal, in particular a human, equine, bovine, porcine, feline, canine, murine, rat, or non-human primate. In preferred embodiments, the subject is a human. The subject may or may not be in need of treatment with a composition of the present disclosure.

### I. Compositions and Microemulsions

The inventors surprisingly found that the addition of a charged polymer complex to a microemulsion resulted in unexpected beneficial features of the charged polymer complex microemulsion. For example, charged polymer complex microemulsions can prevent washout of artificial color in hair. Without being bound by theory, it is thought that the charged polymer microemulsion remains decorated externally with unsatisfied charge, enabling it to bind to sites of polar charged microscopic hair damage, forming a matrix that seals color in. The unexpected features of charged polymer complex microemulsions are distinct from charged microcapsules, which demonstrate no color-retention benefit.

Accordingly, in some aspects, provided herein are compositions comprising a charged polymer complex microemulsion. In some embodiments, the charged polymer complex microemulsion comprises, consists of, or consists essentially of the following components: (a) an oil phase; (b) an aqueous phase; (c) a surfactant; and (d) a charged polymer complex. In some embodiments, the charged polymer complex comprises, consists of, or consists essentially of: one or more amphiphilic anionic charged polymers, one or more amphiphilic cationic charged species, and/or combinations thereof. In some embodiments, the charged polymer complex encapsulates the dispersion phase. In some embodiments, the charged polymer complex forms an interconnected network of fibers.

In another aspect, the inventors surprisingly found that the inclusion of a triglyceride oil in the dilutable microemulsions of the present invention resulted in unexpected beneficial features of the dilutable triglyceride microemulsion. For example, application of the dilutable triglyceride microemulsions of the present invention to food stains on fabrics can effectively remove the food stains from the fabric. Without being bound by theory, it is thought that the triglyceride in the microemulsion acts to solubilize the foods thus making them available for extraction, while the extremely fine scale of the microemulsion enables access into the smallest topological features of the fabric fibers. The result is striking in that adding a food oil to a cleaning composition can paradoxically improve that composition's capacity to clean other food oils. These and other unexpected features of dilutable triglyceride-containing microemulsions are distinct from microemulsions generally, and from surfactants in the absence of the microemulsion structure, which for example do not remove food stains with similar efficacy.

Accordingly, in some aspects, provided herein are compositions comprising dilutable microemulsions of triglycerides and other oils. In some embodiments, the dilutable microemulsion comprises, consists of, or consists essentially of the following components: (a) an oil phase; (b) an aqueous phase; and (c) a surfactant. In some embodiments, the phase comprises, consists of, or consists essentially of: one or more water-immiscible hydrocarbons, one or more silicones, one or more esters, and/or combinations thereof. In some embodiments, the microemulsions are continuously dilutable by 100-fold or more in oil or water without destabilization or changes in appearance.

In another aspect, the inventors surprisingly found that the inclusion of an oil and a surfactant, wherein: the oil has an octanol/water partition coefficient (log Kow) of less than 10; the surfactant HLB is greater than 10; the composition is substantially free of a co-surfactant, a metal halide salt, a hydrotrope, or a combination of two more thereof; the composition is optically transparent; and the composition remains optically transparent upon dilution resulted in unexpected beneficial features. For example, application of the composition of the present invention to food stains on fabrics can effectively remove the food stains from the fabric. Without being bound by theory, it is thought that the composition acts to solubilize the foods thus making them available for extraction. The result is striking in that adding a food oil to a cleaning composition can paradoxically improve that composition's capacity to clean other food oils. In some embodiments, the composition is a triglyceride solubilizer.

Accordingly, in some aspects, provided herein are compositions comprising an oil and a surfactant, wherein: the oil has an octanol/water partition coefficient (log Kow) of less than 10; the surfactant HLB is greater than 10; the composition is substantially free of a co-surfactant, a metal halide salt, a hydrotrope, or a combination of two more thereof; the composition is optically transparent; and the composition remains optically transparent upon dilution resulted in unexpected beneficial features. In some embodiments, the compositions further include an aqueous phase. In some embodiments, the compositions may be a microemulsion.

Accordingly, in some aspects, provided herein are dilutable composition and microemulsions. In some embodiments, the dilutable composition and/or microemulsion comprises, consists of, or consists essentially of the following components: (a) an oil; (b) a surfactant; and optionally (c) an aqueous phase. In some embodiments, the oil comprises, consists of, or consists essentially of: one or more water-immiscible hydrocarbons, one or more silicones, one or more esters, and/or combinations thereof. In some embodiments, the composition and/or microemulsion is continuously dilutable by 100-fold or more in oil or water without destabilization or changes in appearance.

### Charged Polymers and Charged Polymer Complex

In some embodiments, the microemulsions of the present disclosure comprise a charged polymer complex. As defined herein, a charged polymer complex refers to a structure formed by an adduct of a Lewis base and a complementary Lewis acid. In some embodiments, the charged polymer complex comprises a wall and/or cap that encapsulates the dispersion phase of a microemulsion. In some embodiments, the charged polymer complex comprises interconnected polymer fibers. In some embodiments, the charged polymer complex comprises both a wall and/or cap and interconnected fibers. In some embodiments, the charged polymer complex is insoluble.

In some embodiments, the microemulsions of the present disclosure may include a charged polymer component. In some embodiments, the charged polymer may be solubilized primarily in the oil phase. In some embodiments the charged polymer may be solubilized primarily in the water phase. In some embodiments the charged polymer may reside primarily at the interface between the oil phase and the water phase. In some embodiments, the charged polymer is insoluble.

In some embodiments, the charged polymer complex or component comprises one or more amphiphilic charged polymers capable of forming a charged polymer complex or component. In some embodiments, the amphiphilic charged polymer is an anionic polymer. In some embodiments, the amphiphilic charged polymer is a cationic polymer. In some embodiments, the microemulsions comprise at least one amphiphilic anionic polymer and at least one amphiphilic cationic polymer. In some embodiments, one or more of these charged materials may be paired with a complementary charged counterion species.

Amphiphilic anionic polymeric species suitable for use in the microemulsions of the present disclosure include but are not limited to the following Lewis acids and their anions and water-soluble salts: alginic acid, arabic acid, carboxymethylcellulose, carrageenan, saponins, carbomers and related polymers and block copolymers bearing carboxylic acid moieties, collagen, hyaluronic acid, gellan gum, pectin, and generally, polymeric materials of molecular weight above 1000 amu presenting at least two carboxylic acid reactive groups, or combinations of these materials. Additional suitable cationic species are described in U.S. 2008/0138420A1, incorporated by reference herein.

Amphiphilic cationic polymeric species suitable for use in the microemulsions of the present disclosure include the following Lewis bases, anions, and their water-soluble salts: benzalkonium, cetylpyridinium, chitosan, cocodimonium hydroxypropyl hydrolyzed keratin, cocoglucosides hydroxypropyl, hydroxypropyltrimonium hydrolyzed wheat protein, hydroxypropyl oxidized starch PG-trimonium, PEG-3 dioleylamidoethylmonium, laurdimoniumhydroxypropyl decylglucosides, polyquaternium-10, polyquaternium-11, polyquaternium-78, polyquaternium-80, polyquaternium-81, polyquaternium-88, polyquaternium-101, quaternium-79 hydrolyzed silk protein, silicone quaternium-17, silicone quaternium-8, starch hydoxypropyltrimonium, steardimonium hydroxyethylcellulose, steardimonium hydroxypropyl panthenyl PEG-7 dimethicone, cocodimonium hydroxyethylcellulose, polyvinylamine and generally, any water-soluble quaternary amine or similar Lewis base capable of forming a salt with the carboxylic acid moieties of the Lewis acids described above, or combinations of any of these materials. Additional suitable anionic species are described in U.S. 2008/0138420A1.

Counterions suitable for use in the microemulsions of the present disclosure comprise materials that are capable of forming a complex with the amphiphilic anionic polymer or the amphiphilic cationic polymer. Non-limiting examples of counterions include but are not limited to proteins, partially hydrolyzed proteins, charged amino acids, and any of these materials further linked to other compounds that can form salts with the charged polymeric materials (e.g., quaternized hydrolyzed proteins listed in the exemplar Lewis bases above, or quaternary derivatives of these and other proteins or protein fragments). In some embodiments, the counterions impart particular biological compatibility, additional reactive sites, or other desirable properties to the charged polymer complex or component. For example, a simple aqueous solution of approximately 1% hydrolyzed rice protein solids readily forms visible precipitates in the presence of sufficient polyquaternium-10, and this material therefore can be incorporated into the materials of the present disclosure and participate in charge-complexation. In some embodiments, hydrolyzed proteins are useful in forming charge-complexation. Non-limiting examples of hydrolyzed proteins include commercially available hydrolysates of keratin, collagen, silk, rice, soy, wheat, and milk. In some embodiments, similarly charged amino acids or peptides containing at least one charged amino acid can be incorporated into the system of charged counterions of the present disclosure.

In some embodiments, additional polymers with or without ionized sites can be incorporated in the microemulsions of the present disclosure, provided that they can associate with the phase interface. These additional polymers can impart the well-known benefits and qualities of dissolved polymers in microemulsions formulated for pharmaceutical use and/or personal care.

In some embodiments, additional amphiphilic polymers with or without ionized sites can be incorporated in the microemulsions of the present disclosure, provided that they can associate with the phase interface. These additional polymers can impart the well-known benefits and qualities of dissolved amphiphilic polymers in microemulsions formulated for pharmaceutical use and/or personal care.

In some embodiments, the components of the charged polymer complex or component jointly comprise about 0.00001% to about 3%, about 0.00003% to about 2.5%, about 0.00006% to about 2%, about 0.0001% to about 1.5%, about 0.0003% to about 1%, about 0.0006% to about 0.75%, about 0.001% to about 0.5%, about 0.003% to about 0.3%, about 0.006% to about 0.2%, about 0.01% to about 0.1%, or about 0.03% to about 0.06% of the total weight of the charged polymer complex or component microemulsion. In a particular embodiment, the concentration of the components of the charged polymer complex or component jointly comprise is about 0.01% to about 0.1% by weight of the microemulsion.

In some embodiments, the ratio of cationic to anionic components of the charged polymer complex or component is about 1 to 10,000, about 1 to 3,000, about 1 to 1,000, about 1 to 300, about 1 to 100, about 1 to 3, about 3 to 1, about 10 to 1, about 30 to 1, about 100 to 1, about 300 to 1, about 1,000 to 1, about 3,000 to 1, or about 1 to 10,000. In a particular embodiment the ratio of cationic to anionic components of the charged polymer complex or component is about 1 to 10.

In some embodiments, the microemulsion is substantially free of a charged polymer complex. In some embodiments, the microemulsion does not contain a charged polymer complex.

### Uncharged Polymers

In some embodiments, the microemulsions of the present disclosure comprise one or more uncharged polymers. In some embodiments, the concentration of the uncharged polymeric species is about 10% to about 30% by weight of the microemulsion. In some embodiments, the one or more uncharged polymers are selected from the group consisting of agar, acrylic acid, albumins, carrageenans, casein, cellulose gums, including hydroxypropylmethyl cellulose, hydroxypropyl cellulose, and hydroxyethyl cellulose (HEC), methylcellulose and microcrystalline cellulose, chitin derivatives, chondroitin, curdlan, gelatin, dextran, fibrin, fulcelleran, gellan gum, ghatti gum, guar gum, gum tragacanth, heparin, hyaluronic acid, karaya gum, locust bean gum, pea protein, pectin, polyoxyethylene-polyoxypropylene and other synthetic block copolymers, pullulan, starch, soy protein, whey protein, xanthan gum, and zein, polyethylene glycols, polypropylene glycols, poloxamers, poloxamines, polybutylene glycols, polyvinylpyrrolidones, polyvinyl alcohols, polyacrylic acids, polymers of biological organic acids, including poly-lactic acid, poly-lactic co-glycolic acid, starches and starch derivatives, including hydroxypropyl starch, proteins, including partially hydrolyzed proteins, and polypeptides, and combinations and derivatives thereof.

In some embodiments, the charged and uncharged polymers jointly comprise about 0.00001% to about 3%, about 0.00003% to about 2.5%, about 0.00006% to about 2%, about 0.0001% to about 1.5%, about 0.0003% to about 1%, about 0.0006% to about 0.75%, about 0.001% to about 0.5%, about 0.003% to about 0.3%, about 0.006% to about 0.2%, about 0.01% to about 0.1%, or about 0.03% to about 0.06% of the total weight of the microemulsion product. In a particular embodiment, the concentration of the components of the polymers jointly comprise is about 0.01% to about 0.1% by weight of the microemulsion.

In some embodiments, the ratio of charged to uncharged polymer components of the microemulsion is about 1 to 10,000, about 1 to 3,000, about 1 to 1,000, about 1 to 300, about 1 to 100, about 1 to 3, about 3 to 1, about 10 to 1, about 30 to 1, about 100 to 1, about 300 to 1, about 1,000 to 1, about 3,000 to 1, or about 1 to 10,000. In a particular embodiment the ratio of charged to uncharged polymer components of the microemulsion is about 1 to 10.

### Surfactants

The surfactants useful in the compositions and/or microemulsions of the present disclosure include but are not limited to anionic surfactants, cationic surfactants, ionic surfactants, nonionic surfactants, or zwitterionic surfactants. In some embodiments, the surfactant comprises about 0.5% to about 80%, about 1% to about 10%, about 5% to about 20%, about 5% to about 30%, about 5% to about 40%, about 5% to about 50%, about 5% to about 60%, about 5% to about 70%, about 25% to about 75%, about 40% to about 80%, or about 75% to about 90% of the total weight of the compositions and/or microemulsions. In a particular embodiment, the concentration of the surfactant is about 15% to about 40% by weight of the compositions and/or microemulsions.

In some embodiments, the surfactant is selected from the group consisting of: an ethoxylated alcohol represented by the formula R(OC₂H₄)ₙOH, wherein R is a linear, branched, or cyclic alkane moiety; a polyoxyethylene derivative of an ester; a glucoside; an amphiphilic polymeric material, and combinations of two or more thereof. Non-limiting examples of suitable surfactants include: cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, sorbitan oleate decylglucoside crosspolymer (SODC), polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hydroxypropyl methylcellulose, carboxymethylcellulose sodium, methylcellulose, hydroxyethylcellulose, hydroxypropylmethyl-cellulose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctyl sodium sulfosuccinate, dialkylesters of sodium sulfosuccinic acid, sodium lauryl sulfate, alkyl aryl polyether sulfonates, mixtures of sucrose stearate and sucrose distearate, C₁₈H₃₇CH₂C(O)N(CH₃)—CH₂(CHOH)₄(CH₂OH)₂, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, random copolymers of vinyl acetate and vinyl pyrrolidone; cationic lipids, ceteth-25, ceteareth-25, PEG-40 hydrogenated castor oil, PPG-5-Ceteth-20, laureth-7, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, ceteareth-20, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅ dimethyl hydroxyethyl ammonium chloride, C₁₂₋₁₅ dimethyl hydroxyethyl ammonium chloride bromide, C₁₂₋₁₃ pareth 9 (P9), coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₅)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™ ALKAQUAT™, alkyl pyridinium salts; amines, amine salts, amine oxides, imide azolinium salts, protonated quaternary acrylamides, methylated quaternary polymers, and cationic guar.

In accordance with some embodiments, the surfactant is selected from the group consisting of: cetyl pyridinium chloride, phosphatides, cholesterol, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, sorbitan oleate decylglucoside crosspolymer (SODC), polyethylene glycols, dodecyl trimethyl ammonium bromide, polyoxyethylene stearates, phosphates, sodium dodecylsulfate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctyl sodium sulfosuccinate, dialkylesters of sodium sulfosuccinic acid, sodium lauryl sulfate, alkyl aryl polyether sulfonates, mixtures of sucrose stearate and sucrose distearate, C₁₈H₃₇CH₂C(O)N(CH₃)—CH₂(CHOH)₄(CH₂OH)₂, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, random copolymers of vinyl acetate and vinyl pyrrolidone; cationic lipids, ceteth-25, ceteareth-25, PEG-40 hydrogenated castor oil, PPG-5-Ceteth-20, laureth-7, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, ceteareth-20, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅ dimethyl hydroxyethyl ammonium chloride, C₁₂₋₁₅ dimethyl hydroxyethyl ammonium chloride bromide, C₁₂₋₁₃ pareth 9 (P9), coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, Cis trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, polyquaternium-10, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™ ALKAQUAT™, alkyl pyridinium salts; amines, amine salts, amine oxides, imide azolinium salts, protonated quaternary acrylamides, methylated quaternary polymers, and cationic guar.

In particular embodiments, the surfactant is selected from ethoxylated alcohols and phenols of the form R(OC₂H₄)ₙOH, where R comprises a linear, branched, or cyclic alkane moiety or a combination of such components. Other suitable materials include polyoxyethylene derivatives of esters, glucosides, for example Heptyl Glucoside (Sepiclear G7, Seppic, Florham Park, N.J.), and amphiphilic polymeric materials, for example Pluronic F68 (BASF, Fairfield, N.J.) and Sorbitan Oleate Decylglucoside Crosspolymer (PolySuga® Mulse D6, Colonial Chemical, South Pittsburgh, Tenn.) hereafter termed SODC. Some ethoxylates that are liquids at ambient temperature are of particular utility in regard to reducing the heat input required to form the dispersed systems of the present disclosure, for example those formed from fatty alcohols with fewer than 16 carbons. Other ethoxylated materials that are room temperature solids, such as those formed from cetyl and stearyl alcohol are of particular utility due to their wide availability from inexpensive and renewable plant sources. In some embodiments, the surfactant is selected from the group consisting of: ceteareth-20, ceteth-25, ceteareth-25, PEG-40 hydrogenated castor oil, PPG-5-ceteth-20, laureth-7, PEG-40 stearate, SODC, P9, and combinations of two or more thereof. In some embodiments, the surfactant is selected from the group consisting of: SODC, P9, and combinations thereof. In some embodiments, the compositions and/or microemulsions include one or more additional surfactants. Other surfactants may be desirable due to consumer preferences for products not containing ethoxylates. In some embodiments, the surfactant is dissolved with the oil to form a visually clear mixture prior to addition of water, although this is not an explicit requirement.

In some embodiments, more than one surfactant is used in the charged polymer complex microemulsion. For example, a combination of two, three, four, five, six, seven, eight, nine, ten, or more surfactants is used in the charged polymer complex microemulsion.

In some embodiments, more than one surfactant is used in the dilutable microemulsion. For example, a combination of two, three, four, five, six, seven, eight, nine, ten, or more surfactants is used in the dilutable microemulsion.

Surprisingly, the microemulsions of the present disclosure do not require that the surfactant alone form clear aqueous solutions. For example, dilution of 5 parts bottle-strength (nominally 62% solids) SODC in 30 parts water forms a strongly hazy, non-clear dispersion even after heating to 80° C. and cooling to 25° C. Yet the same quantity of SODC may be combined with 1 part capric/caprylic triglycerides (Lexol GT, Inolex Chemical Co., Philadelphia, Pa.), hereafter CTG, to form a clear solution (about 30 ntu). When this SODC/CTG solution is combined with heating to 80° C. and cooled to 25° C., an example of the clear microemulsions of the present disclosure is formed.

In some embodiments, the hydrophilic-lipophilic-balance (HLB) formalism does not strongly correlate with the set of surfactants suitable for use in the present disclosure (demonstrated examples of suitable surfactants range over 8 HLB units). However, in some embodiments, successful surfactants used in the present disclosure possess HLB of 8 or higher.

In some embodiments, one or more of the surfactants may not be directly oil soluble, and may be provided commercially as a concentrated solution in water. Thus, in some embodiments, some water may be present in the surfactant.

Non-limiting examples of surfactants that form continuously dilutable microemulsions of the present disclosure with 1:10 mixtures of argan oil and CTG include: ceteareth-20 (Eumulgin B2, BASF, Florham Park, N.J.), ceteth-25 (PEL-ALC CA-25) and/or ceteareth-25 (PEL-ALC CSA-25) (Elé Corp., McCook, Ill.), PEG-40 hydrogenated castor oil (HC-40, Hallstar, Chicago, Ill.), PPG-5-Ceteth-20 (AM-Acquasolve, Chemyunion, Union, N.J.), laureth-7 (Genopol LA 070, Clariant, Charlotte, N.C.), PEG-40 Stearate (Myrj 52, Uniqema, New Castle, Del.), and SODC and P9.

### Oil/Oil-Phase

The oil/oil-phase comprises, consists of, or consists essentially of one or more oils suitable for use in the microemulsions of the present disclosure. Such oils include but are not limited to: esters, including glycerides, fats, waxes, terpenes, and any largely water-immiscible materials of intermediate polarity. In some embodiments, the oil is a water-immiscible oil or an oil that is characterized by low water immiscibility.

In some embodiments, when an aqueous phase is present, the oil phase comprises about 0.01% to about 50%, about 0.01 to about 0.1%, about 0.01 to about 1%, about 0.01 to about 10%, about 0.1% to about 10%, about 5% to about 20%, about 5% to about 30%, about 5% to about 40%, about 5% to about 50%, about 5% to about 60%, about 1% to about 5%, about 2% to about 7%, about 5% to about 15%, or about 15% to about 40% of the total weight of the composition and/or microemulsion. In a particular embodiment, the concentration of the oil phase is about 0.1% to about 10% by weight of the composition and/or microemulsion.

In some embodiments, the oil/oil phase comprises, consists of, or consists essentially of one or more plant- or mineral-based glycerides, fats, and/or waxes including, but not limited to: almond oil, apricot kernel oil, argan nut oil, avocado oil, babassu oil, baobab oil, black cumin oil, borage oil, broccoli seed oil, beeswax, camelina oil, camellia seed oil, canola oil, carrot seed oil, castor oil, chia seed oil, citrus oils, cocoa butter, coconut oil, CTG, cranberry seed oil, daikon seed oil, evening primrose oil, flax seed oil, grape seed oil, hazelnut oil, hemp seed oil, jojoba oil, candellila wax, carnauba wax, ozokerite, paraffin, stearin, kokum butter, kukui nut oil, lanolin, macadamia nut oil, mango butter, marula oil, meadowfoam seed oil, medium chain triglycerides, monoi oil, moringa oil, neem oil, olive oil, palm fruit oil, palm kernel oil, pomegranate seed oil, prickly pear seed oil, pumpkin seed oil, red palm oil, raspberry seed oil, rice bran oil, rosehip oil, sacha inchi oil, safflower oil, seabuckthorn fruit oil, sesame seed oil, shea nut oil, shorea butter, soybean oil, strawberry seed oil, sunflower oil, tamanu oil, walnut oil, and wheat germ oil, and chemically modified, esterified, de-esterified or hydrogenated forms of such materials. In some embodiments, the oil/oil phase comprises a-pinene, camphene, b-pinene, sabinene, myrcene, a-terpinene, linalool, b-bisabolene, limonene, trans-a-bergamotene, nerol neral, or a combination of two or more thereof. In some embodiments, the oil/oil phase comprises, consists of, or consists essentially of one or more of almond oil, apricot kernel oil, argan oil, avocado oil, babassu oil, baobab oil, black cumin oil, borage oil, broccoli seed oil, beeswax, C₁₂₋₁₅ alkyl benzoate, buruti oil, camelina oil, camellia seed oil, canola oil, capric/caprylic triglycerides (CTG), carrot seed oil, castor oil, chia seed oil, citrus oil, cocoa butter, coconut oil, cranberry seed oil, daikon seed oil, evening primrose oil, flax seed oil, grape seed oil, hazelnut oil, hemp seed oil, jojoba oil, kokum butter, kukui nut oil, lanolin, macadamia nut oil, mango butter, marula oil, meadowfoam seed oil, medium chain triglycerides (MCT), monoi oil, moringa oil, neem oil, octyl methoxycinnamate, octocrylene, olive oil, palm fruit oil, palm kernel oil, pomegranate seed oil, prickly pear seed oil, pumpkin seed oil, red palm oil, raspberry seed oil, rice bran oil, rosehip oil, sacha inchi oil, safflower oil, seabuckthorn fruit oil, sesame seed oil, shea nut oil, shorea butter, soybean oil, strawberry seed oil, sunflower oil, tamanu oil, walnut oil, wheat germ oil, and chemically modified, esterified, partially de-esterified or hydrogenated forms thereof, and esters of organic acids, including acetic, propionic, butyric, valeric, caproic, lactic, malic, citric and benzoic acid esters, and simple esters of fatty acids. In some embodiments, the oil/oil-phase includes citrus oil (e.g., lemon peel oil).

In some embodiments, esters varying from high to low polarity and correspondingly different partition coefficients are useful oil phases in the present disclosure. In a non-limiting example, 1 part triethyl citrate (Citroflex 2, Vertellus Performance Materials, Greensboro, N.C.) with K_{Pow}=1.17, combined with 9 parts P9 may be diluted with any volume of water without loss of clarity. Similarly acetyl tributyl citrate (Citroflex A4, Vertellus Performance Materials, Greensboro, N.C.) with K_{Pow}=6.9, can be substituted for triethyl citrate with no loss of clarity at any dilution.

Emulsion and microemulsion systems are widely understood to require matching of HLB values between surfactants and different dispersed phases, and generally dispersed phases of intermediate polarity such as triglycerides have different optimal HLB requirements than for example dispersed phases of very low polarity like silicones or alkanes. It was therefore very surprising to discover that the systems of the present disclosure can be readily formed with oil phases consisting of silicones and alkanes, as well as a variety of organosilicone materials.

For example, a system comprising 10 parts of D9 surfactant and 1 part hexamethyl disiloxane (DC-200 0.65 CST, Dow Corning, Midland, Mich.) forms an infinitely dilutable, water clear microemulsion of the type taught in the present disclosure. Similarly, substitution of Eicosane (Permethyl 102A, Presperse Corp, Somerset, N.J.), a mixture of isohexadecane, isododecane and C₁₃₋₁₅ alkanes (SiClone SR-5, Presperse), or a mixture of C14-22 alkanes (Lilac, Sonneborne Inc., Parsippany, N.J.) for the hexamethyl disiloxane produces a similar water-clear product. Other exemplary materials that form the dilutable transparent microemulsions of the present disclosure include without limitation, phenyltrimethicone (SPI-PTM, Silicones Plus), C15-19 alkanes (Emogreen L15, Seppic Air Liquide, Houston, Tex.), liquefied butane and propane (in confined space), octamethyl trisiloxane (Q7-9180 Silicone Fluid, Dow Corning), octamethyl cyclotetrasiloxane (344 Fluid, Dow Corning), amodimethicone (KF-8005, Shin-Etsu Corp., Newark, Calif.), trimethyl-terminated aminopropyl phenylsequisiloxane (2-2078 Fluid, Dow Corning), and other silicone ethers, including without limitation, dimethylpolysiloxanes, polyether-modified silicones, amino-modified silicones, carboxy-modified silicones, methylphenyl silicone, fatty acid-modified silicones, alcohol-modified silicones, aliphatic alcohol-modified silicones, epoxy-modified silicones, fluorine-modified silicones, cyclic silicones, alkyl-modified silicones, with chain length ranging from 2 to 25,000 SiO subunits. Polyisobutylene, (TPC 175, TPC Group, Houston, Tex.) and derivatives of polyisobutylene, such as hydrogenated polyisobutylene (Permethyl 103a, Presperse) can also be used as the oil component of the present disclosure. Substitution of an alkane hydrogen group with, for example, chlorine, does not alter the suitability of the alkane. For example, methylene chloride, chloroform, and tetrafluoroethane are all readily solubilized according to the present disclosure. Generally, alkanes and alkane derivatives, including substituted, cyclic and branched alkanes, with carbon chain lengths ranging from 1 to 25,000 are understood to suitable for use in the present disclosure.

In some embodiments, the oil phase may comprise more than one oil component. For example, oils may be used singly or in combination in the systems of the present disclosure. If more than one oil is used, the component oils may be fully miscible, partially miscible, or essentially fully immiscible. If fully immiscible oils are used, at least one oil must form the dilutable microemulsions of the present disclosure. In some embodiments, one oil (“X”) may form a microemulsion as taught herein, while at least one additional oil (“Y”) may not independently form such a microemulsion. In some such cases, in which X and Y are miscible, Y's solubility in X may result in the combination X+Y being suitable to form the microemulsion systems of the present disclosure, similar to X alone.

For example, hexamethyl disiloxane readily forms the microemulsions of the present disclosure, while a 100 CSt dimethicone (SPI-100, Silicones Plus) does not. However, the two silicones are fully miscible, and 1 part 100 CSt dimethicone, 10 parts hexamethyl disiloxane, 100 parts D9 surfactant, and 300 parts water combine to form a microemulsion according to the present disclosure. A useful and interesting property of the system thus formed is that while this system is stable with regard to separation or coalescence, if the product thus formed is open to air such that the volatile hexamethyl disiloxane component can evaporate, the system loses stability, and the dimethicone component coalesces to form a macroscopic emulsion. This evaporative instability is of particular utility when such a system is applied to a surface, such that in microemulsion form the components can collectively wet into and penetrate very fine morphological features, but upon inhomogeneous evaporative loss, the microemulsion becomes unstable and can deposit larger droplets of the dimethicone component into features that would ordinarily be inaccessible to larger droplets. This behavior can be generalized across any of the systems of the present disclosure in which either the aqueous or the oil phase contains one or more components that may be disproportionately removed by for example, evaporation, chemical reaction, photonic processes, thermal or other processes by which one component may be selectively depleted so as to render the microemulsion unstable and prone to coalescence. For example, a microemulsion of an oil system comprising a volatile component such as hexamethyl disiloxane and a benefit agent such as a silicone that does not readily form the microemulsions of the present disclosure independently, can be applied to a surface, such as human hair, and upon brief exposure to air evaporative loss of the volatile component can destabilize the microemulsion resulting in coalescence and associated enhanced deposition of the non-volatile silicone component onto the hair, such that deposition of that component accesses finer morphological features than possible with a standard macroemulsion and such that removal of that component by the surfactant of the present disclosure is inhibited, as its capacity to finely disperse that component has been exceeded.

In particular embodiments of the microemulsion, the oil phase comprises one or more of: argan oil, CTG, octyl methoxycinnamate, and octocrylene.

### Aqueous Phase

In some embodiments, the aqueous phase comprises, consists of, or consists essentially of water. In some embodiments, the water is deionized (DI) water, distilled water, or distilled, deionized (DD) water. In some embodiments, the aqueous phase comprises about 10% to about 99.9%, about 10% to about 80%, about 30% to about 95%, about 20% to about 70%, about 75% to about 95%, about 80% to about 99.9%, about 50% to about 99.9%, about 50% to about 95%, about 25% to about 75%, about 75% to about 85%, or about 15% to about 60% of the total weight of the charged polymer complex microemulsion. In a particular embodiment, the concentration of the aqueous phase is about 50% to about 80% by weight of the microemulsion.

### Exclusions

In some embodiments, the compositions and/or microemulsions of the present disclosure do not comprise a co-surfactant. As used herein, a “co-surfactant” is a chemical moiety that when used in combination with a surfactant, further reduces the surface tension of a liquid. Nonlimiting examples of co-surfactants include diethylene glycol monoethyl ether; 2-(2-Ethoxyethoxy)ethanol, glycerin, ethylene glycol, propylene glycol, ethanol, and propanol. In particular embodiments, the microemulsions of the present disclosure do not comprise a glycol.

In some embodiments, the compositions and/or microemulsions of the present disclosure do not comprise a metal halide salt. In some embodiments, the compositions and/or microemulsions of the present disclosure do not comprise a hydrotope. Non-limiting examples of hydrotopes include salts of toluene sulfonic acid, xylene sulfonic acid, and cumene sulfonic acid.

Suitable Ratios of Composition and/or Microemulsion Components

Non-limiting, exemplary ratios of each component of the compositions and/or microemulsions of the present disclosure are provided in Table 1 below. The numbers in the ratios represent the percentage weight of each component by weight of total composition. For example, in row 1, the table presents a ratio of (a) oil phase: (b) aqueous phase: (c) surfactant: (d) anionic polymer: (e) cationic polymer of 1%:54%:40%:5%:0%.

TABLE 1
Exemplary Ratios

| | Microemulsion Component | | | | |
|---|---|---|---|---|---|
| | | | | (d) | (e) |
| Exemplary | | (b) | | amphiphilic | amphiphilic |
| Ratio | (a) | aqueous | (c) | anionic charged | cationic charged |
| (out of 100) | oil phase | phase | surfactant | polymer | polymer |
| 1 | 1 | 78.98 | 20 | 0.01 | 0.01 |
| 2 | 10 | 48 | 40 | 1 | 1 |
| 3 | 20 | 36 | 40 | 2 | 2 |
| 4 | 20 | 37.9 | 40 | 2 | 0.1 |
| 5 | 20 | 37.9 | 40 | 0.1 | 2 |
| 6 | 79.98 | 1 | 20 | 0.01 | 0.01 |
| 7 | 40 | 48 | 10 | 1 | 1 |
| 8 | 40 | 39.98 | 20 | 0.01 | 0.01 |
| 9 | 40 | 37.9 | 20 | 2 | 0.1 |
| 10 | 40 | 37.9 | 20 | 0.1 | 2 |
| 11 | 10 | 49.89 | 40 | 0.1 | 0.01 |
| 12 | 10 | 49.89 | 40 | 0.01 | 0.1 |
| 13 | 0.1 | 98.88 | 1 | 0.01 | 0.01 |
| 14 | 0.01 | 99.888 | 0.1 | 0.001 | 0.001 |
| 15 | 0.001 | 99.9888 | 0.01 | 0.001 | 0.001 |

Additional non-limiting, exemplary ratios of each component of the compositions and/or microemulsions of the present disclosure are provided in Table 2 below. The numbers in the ratios represent the percentage weight of each component by weight of total composition. For example, in row 1, the table presents a ratio of (a) oil phase: (b) aqueous phase: (c) surfactant: (d) charged polymer: (e) uncharged polymer of 1%:54%:40%:5%:0%.

TABLE 2
Additional Exemplary Ratios

| | Microemulsion Component | | | | |
|---|---|---|---|---|---|
| | | | | (d) | (e) |
| Exemplary | | (b) | | amphiphilic | amphiphilic |
| Ratio | (a) | aqueous | (c) | charged | uncharged |
| (out of 100) | oil phase | phase | surfactant | polymer | polymer |
| 1 | 1 | 78.98 | 20 | 0.01 | 0.01 |
| 2 | 10 | 48 | 40 | 1 | 1 |
| 3 | 20 | 36 | 40 | 2 | 2 |
| 4 | 20 | 37.9 | 40 | 2 | 0.1 |
| 5 | 20 | 37.9 | 40 | 0.1 | 2 |
| 6 | 79.98 | 1 | 20 | 0.01 | 0.01 |
| 7 | 40 | 48 | 10 | 1 | 1 |
| 8 | 40 | 39.98 | 20 | 0.01 | 0.01 |
| 9 | 40 | 37.9 | 20 | 2 | 0.1 |
| 10 | 40 | 37.9 | 20 | 0.1 | 2 |
| 11 | 10 | 49.89 | 40 | 0.1 | 0.01 |
| 12 | 10 | 49.89 | 40 | 0.01 | 0.1 |
| 13 | 0.1 | 98.88 | 1 | 0.01 | 0.01 |
| 14 | 0.01 | 99.888 | 0.1 | 0.001 | 0.001 |
| 15 | 0.001 | 99.9888 | 0.01 | 0.001 | 0.001 |

Additional non-limiting, exemplary ratios of the surfactant to oil include weight ratios from about 4:1 to about 20:1. In some embodiments, the weight ratio may be from about 4:1 to about 15:1, about 4:1 to about 12:1, or about 4:1 to about 10:1. In some embodiments, the weight ratio may be from about 6:1 to about 20:1, about 10:1 to about 20:1, or about 13:1 to about 20:1.

### II. Methods of Manufacturing Microemulsions

### Charged Polymer Complex Microemulsions

In some aspects, provided herein are methods of manufacturing charged polymer complex microemulsions. In some embodiments, the charged polymer complex microemulsions of the present disclosure are manufactured by a method comprising admixing the following components: (a) a substantially water-immiscible oil phase; (b) an aqueous phase; (c) a surfactant; and (d) one or more amphiphilic anionic charged polymers. In other embodiments, the microemulsions of the present disclosure are formed by admixing the following components: (a) a substantially water-immiscible oil phase; (b) an aqueous phase; (c) a surfactant; (d) one or more amphiphilic anionic charged polymers; and (e) one or more amphiphilic cationic charged species.

In some embodiments of the method, the components are admixed simultaneously, sequentially, or consecutively. In some embodiments of the method, a subset of the components are pre-mixed, and then combined with the remaining components. For example, in one embodiment, the microemulsion of core material in the present method is performed independent of the charged polymer complex-forming step: Components (a) and (c) are admixed until a visually clear solution is formed, with no striation visible upon swirling of the liquid. Component (b) is then added to the mixture of (a) and (c), optionally pre-warmed to 60° C. or higher to speed incorporation. In some embodiments, the non-aqueous phase thickens dramatically upon the addition of water but remains largely clear. Admixing continues with visible mixing striation but without the opaque appearance of an emulsion. During admixing, wavelength-dependent scattering may be observed with minor haze, but upon completion of mixing, the product is sufficiently clear. Finally, components (d) and/or (e) are added to the mixture, simultaneously or consecutively.

In other embodiments, components (a) and (c) are admixed prior to the addition of components (b), (d), and optionally (e). In some embodiments, (b) and (c) are admixed prior to the addition of components (a), (d), and optionally (e). In some embodiments, (a), (b), (c), and (d) are admixed prior to the addition of component (e). In other embodiments, (a), (b), (c), and (e) are admixed prior to the addition of component (d). In some embodiments, (d) or (e) is admixed with the aqueous phase prior to forming the microemulsion.

In some embodiments, the present method does not require complete solvation of components (d) or (e) in the dispersion phase. Therefore, a wide variety of core materials may be potentially captured by the charged polymer complex of the disclosed microemulsion. Because the emulsion step may be performed independent of the charged polymer complex-forming step, the disclosed method affords greater flexibility with regard to the methods and timing of the emulsion step. Accordingly greater ease of manufacturing is provided by the disclosed method.

In some embodiments, components (d) and (e) of the microemulsion are complementary in that one is a Lewis acid and the other is a Lewis base and together they react to form an insoluble Lewis acid-Lewis base salt. As used herein the word “complementary” refers to Lewis acids and Lewis bases that react to form insoluble salts. Either (e) (a Lewis acid reactant) or (d) (a Lewis base reactant) may be used as the first charged polymer complex reactant dissolved in the continuous phase in which the core material is dispersed. In some embodiments, the Lewis base reactant is used because many of these compounds are also effective emulsifying agents. By contrast, aqueous solutions of suitable Lewis acid compounds may or may not readily emulsify the core material.

In accordance with some embodiments, the oil phase (a) is admixed with the surfactant phase (c), and the to the resulting mixture is added in sequence, the aqueous phase (b), anionic phase (d), and cationic phase (e). In some embodiments, the anionic phase (d) is added after the cationic phase (e). In some embodiments, the aqueous phase (b) is admixed with the surfactant phase (c), and the to the resulting mixture is added in sequence, the oil phase (d), anionic phase (d), and cationic phase (e). In some embodiments, the aqueous phase (b) is admixed with the surfactant phase (c), and the to the resulting mixture is added in sequence, anionic phase (d), cationic phase (e), and the oil phase (d). In some embodiments, the oil, aqueous, and surfactant phases, (a), (b), and (c) are admixed simultaneously, followed by the addition of the anionic phase (d), and then the cationic phase (e). In some embodiments, the oil, aqueous, and surfactant phases, (a), (b), and (c) are admixed simultaneously, followed by the addition of the cationic phase (e), and then the anionic phase (d).

Admixing to form the microemulsion can be performed by a method comprising one or more of: combining, mixing, nutation, shaking, agitation, and/or stirring the components. In some embodiments, admixing is limited to methods that produce low shear stress on the composition. In particular embodiments, admixing does not produce high shear stress on the composition. In some embodiments, the microemulsions of the present disclosure are spontaneous or self-forming, meaning that the method of manufacture does not comprise use of high energy mechanical input. High energy mechanical input includes but is not limited to sonication at ultrasonic frequencies greater than or equal to 20 kHz and high shear homogenization such as produced by rotor-stator homogenizers and piston-type homogenizers.

Without wishing to be bound by any particular theory or explanation, it is thought that forces of polar solvent interaction drive the lipophilic end of the amphiphilic charged polymer to solvate into the less-polar interior of the dispersed phase, leaving the hydrophilic moieties solvated in the aqueous phase and thus causing the reactant to preferentially accumulate at the dispersion or droplet surface. Thus it is believed that the amphiphilic charged polymer tends to rapidly collect at the droplet-continuous phase interface as the microemulsion is formed, stabilizing the microemulsion, and, in some embodiments, providing a reaction site for the amphiphilic cationic charged species.

Accordingly, in some embodiments, manufacture of the microemulsions of the present disclosure does not require the use of a co-surfactant. As used herein, a “co-surfactant” is a chemical moiety that when used in combination with a surfactant, further reduces the surface tension of a liquid. Nonlimiting examples of co-surfactants include diethylene glycol monoethyl ether; 2-(2-Ethoxyethoxy)ethanol, glycerin, ethylene glycol, propylene glycol, ethanol, and propanol. In some embodiments, the manufacture of the microemulsions of the present disclosure does not require use of a glycol.

In some embodiments, manufacture of the microemulsions of the present disclosure does not require the use of a metal halide salt.

Encapsulated forms of the charged polymer complex microemulsions of the present disclosure are formed by inclusion of one or more charged amphiphilic polymers in the system and their complexation with counterions to form precipitates at immiscible phase interfaces. In some embodiments, specific surfactant materials may participate in such ionic interactions.

In some embodiments of the method of manufacturing microemulsions, the microemulsions or microemulsion components are admixed at a temperature between about 4° C. to about 25° C. In other embodiments, the method further comprises heating the system to facilitate dissolution. In some embodiments, the method further comprises heating one or more components prior to admixing. In some embodiments, the microemulsions are manufactured at a temperature between about 20° C. to about 30° C. In some embodiments, the microemulsions are manufactured at a temperature between about 20° C. to about 50° C. In some embodiments, the microemulsions are manufactured at a temperature between about 30° C. to about 75° C. In some embodiments, the microemulsions are manufactured at a temperature between about 50° C. to about 90° C. In some embodiments, the microemulsions are manufactured at a temperature between about 75° C. to about 95° C. In some embodiments, the microemulsions are manufactured at a temperature less than the boiling point of water, about 100° C.

In some embodiments, the microemulsions are prepared without the use of alcohol or glycols, salts, or other linkers that would interfere with foaming if used in cleansing formulations. For example, ordinary emulsions or dispersions of oils tend to inhibit foaming properties of surfactant systems. The charged polymer complex microemulsions of the present disclosure permit incorporation of oils into optically clear cleansing formulations without inhibition of foam, and have been found to be synergistic in such systems, boosting foam volume and production. Other embodiments of the present disclosure provide fine dispersions that display some optical haze (wavelength-dependent scatter), but are so finely divided as to be stable in storage under a variety of conditions, resisting stratification or separation despite the absence of any added stabilizing agents.

### Dilutable Microemulsions

In some aspects, provided herein are methods of manufacturing dilutable microemulsions. In some embodiments, the dilutable microemulsions of the present disclosure are manufactured by a method comprising admixing the following components: (a) a substantially water-immiscible oil phase; (b) an aqueous phase; (c) a surfactant; and optionally (d) one or more amphiphilic polymers. In other embodiments, the microemulsions of the present disclosure are formed by admixing the following components: (a) a substantially water-immiscible oil phase; (b) an aqueous phase; (c) a surfactant; and optionally (d) one or more amphiphilic polymers; and optionally (e) one or more amphiphilic charged species.

In some embodiments of the method, the components are admixed simultaneously, sequentially, or consecutively. In some embodiments of the method, a subset of the components are pre-mixed, and then combined with the remaining components. For example, in one embodiment, the microemulsion of core material in the present method is performed independent of the step in which polymers may be added, as described in the following sequence. 1. Components (a) and (c) are admixed until a visually clear solution is formed, with no striation visible upon swirling of the liquid. 2. Component (b) is then added to the mixture of (a) and (c), optionally pre-warmed to 60° C. or higher to speed incorporation. In some embodiments, the non-aqueous phase thickens dramatically upon the addition of water but remains largely clear. Admixing continues with visible mixing striation but without the opaque appearance of an emulsion. During admixing, wavelength-dependent scattering may be observed with minor haze, but upon completion of mixing, the product is sufficiently clear. 3. Finally, components (d) and/or (e) are added to the mixture, simultaneously or consecutively.

In other embodiments, components (a) and (c) are admixed prior to the addition of components (b), (d), and optionally (e). In some embodiments, (b) and (c) are admixed prior to the addition of components (a), (d), and optionally (e). In some embodiments, (a), (b), (c), and (d) are admixed prior to the addition of component (e). In other embodiments, (a), (b), (c), and (e) are admixed prior to the addition of component (d). In some embodiments, (d) or (e) is admixed with the aqueous phase prior to forming the microemulsion.

In some embodiments, the present method does not require complete solvation of components (d) or (e) in the dispersion phase. Therefore, a wide variety of dispersed materials may be potentially included in the disclosed microemulsion. Because the systems of the present invention produce nanometer scale intermiscibility of the normally immiscible oil and water phases, and because the systems form equally well in any order of addition, and because the systems form without any requirement for high shear or expensive equipment, the disclosed method affords greater flexibility with regard to the methods and timing of forming emulsions as compared to standard emulsification processes. Accordingly greater ease of manufacturing is provided by the disclosed method.

In accordance with some embodiments, the oil phase (a) is admixed with the surfactant phase (c), and the to the resulting mixture is added the aqueous phase. In some embodiments, the aqueous phase (b) is admixed with the surfactant phase (c), and the to the resulting mixture is added in sequence, the oil phase. In some embodiments, the oil, aqueous, and surfactant phases, (a), (b), and (c) are admixed simultaneously. In some embodiments, one or more of the oil, aqueous, and/or surfactant phases, (a), (b), and (c), are combined with at least one polymer or active agent (d) prior to being combined to form the microemulsion. In some embodiments at least one polymer or active agent is added to the microemulsion after formation. In some embodiments the polymer or active agent is added to a concentrated microemulsion prior to dilution. In some embodiments the polymer or active agent is added to a microemulsion after dilution.

Admixing to form the microemulsion can be performed by a method comprising one or more of: combining, mixing, nutation, shaking, agitation, and/or stirring the components. In some embodiments, admixing is limited to methods that produce low shear stress on the composition. In particular embodiments, admixing does not produce high shear stress on the composition. In some embodiments, the microemulsions of the present disclosure are spontaneous or self-forming, meaning that the method of manufacture does not comprise use of high energy mechanical input. High energy mechanical input includes but is not limited to sonication at ultrasonic frequencies greater than or equal to 20 kHz and high shear homogenization such as produced by rotor-stator homogenizers and piston-type homogenizers.

Without wishing to be bound by any particular theory or explanation, it is thought that forces of polar solvent interaction drive the lipophilic end of the amphiphilic charged polymer to solvate into the less-polar interior of the dispersed phase, leaving the hydrophilic moieties solvated in the aqueous phase and thus causing the polymer to preferentially accumulate at the dispersion or droplet surface. Thus it is believed that the amphiphilic charged polymer tends to rapidly collect at the droplet-continuous phase interface as the microemulsion is formed, stabilizing the microemulsion.

Accordingly, in some embodiments, manufacture of the microemulsions of the present disclosure does not require the use of a co-surfactant. As used herein, a “co-surfactant” is a chemical moiety that when used in combination with a surfactant, further reduces the surface tension of a liquid. Nonlimiting examples of co-surfactants include diethylene glycol monoethyl ether; 2-(2-Ethoxyethoxy)ethanol, glycerin, ethylene glycol, propylene glycol, ethanol, and propanol. In some embodiments, the manufacture of the microemulsions of the present disclosure does not require use of a glycol.

In some embodiments, manufacture of the microemulsions of the present disclosure does not require the use of a metal halide salt.

In some embodiments of the method of manufacturing microemulsions, the microemulsions or microemulsion components are admixed at a temperature between about 4° C. to about 25° C. In other embodiments, the method further comprises heating the system to facilitate dissolution. In some embodiments, the method further comprises heating one or more components prior to admixing. In some embodiments, the microemulsions are manufactured at a temperature between about 20° C. to about 30° C. In some embodiments, the microemulsions are manufactured at a temperature between about 20° C. to about 50° C. In some embodiments, the microemulsions are manufactured at a temperature between about 30° C. to about 75° C. In some embodiments, the microemulsions are manufactured at a temperature between about 50° C. to about 90° C. In some embodiments, the microemulsions are manufactured at a temperature between about 75° C. to about 95° C. In some embodiments, the microemulsions are manufactured at a temperature less than the boiling point of water, about 100° C.

In some embodiments, the microemulsions are prepared without the use of alcohol or glycols, salts, or other linkers that would interfere with foaming if used in cleansing formulations. For example, ordinary emulsions or dispersions of oils tend to inhibit foaming properties of surfactant systems. The microemulsions of the present disclosure permit incorporation of oils into optically clear cleansing formulations without inhibition of foam, and have been found to be synergistic in such systems, boosting foam volume and production. Other embodiments of the present disclosure provide fine dispersions that display some optical haze (wavelength-dependent scatter), but are so finely divided as to be stable in storage under a variety of conditions, resisting stratification or separation despite the absence of any added stabilizing agents.

### Dilution the of the Microemulsion

In some embodiments, the microemulsions of the present disclosure are self-diluting in water and/or infinitely dilutable in water without loss of clarity. Remarkably, it was found that the optical clarity of the disclosed microemulsions is retained regardless of the total amount of water added, although some haze may be apparent during mixing to uniformity. That this water-clarity is maintained throughout a wide range of water concentrations (from 0.000001% to 1,000,000% dilution of water) and to much larger dilution volumes demonstrates that dilution of this system does not result in a non-miscible phase. In some embodiments, heating may facilitate dissolution of the materials to generate the clear system. In some embodiments, the clarity of the system is not temperature-dependent between about 0° C. and up to about 50° C. and higher, temperatures normally considered extremes for consumer products. Notably, freezing and re-thawing do not cause any visible changes in typical products of the present disclosure.

In another embodiment, the charged polymer complex microemulsions are infinitely dilutable with the oil phase without loss of clarity. Generally, if the microemulsion of the present disclosure can be formed to be water-dilutable, a symmetrically inverted product can be formed by dilution with the oil phase.

### Drying Behavior

In some embodiments, the microemulsion systems of the present disclosure can be dried via evaporation of the water continuous phase, resulting in a stable oil/surfactant/charged polymer complex solution. In some embodiments, the dilutable microemulsion systems of the present disclosure can be dried via evaporation of the water continuous phase, resulting in a stable oil/surfactant solution. These dried solutions are capable of being re-hydrated with water in any proportion to re-form the clear systems described herein. Without being bound by theory, when microemulsions are dried upon a substrate material, it is believed that the oil phase is deposited onto the material, along with the surfactant. In some embodiments, the oil phase may be essentially non-interacting with the surface. In other embodiments, the oil phase may dissolve materials already present on the surface. In yet other embodiments the oil phase may be partially absorbed or dissolved into the surface. In some embodiments, the oil phase may be non-interacting with some components of the surface, but strongly interacting with other components of the surface.

In some embodiments, re-solubilization of the microemulsion system: (i) removes the deposited oil/surfactant/charged polymer complex system, and/or (ii) removes other materials solubilized by the oil/surfactant/charged polymer complex system, and/or (iii) removes only part of the oil/surfactant/charged polymer complex system originally deposited, or (iv) exhibits combinations of these behaviors. Because of these features, the types of surfaces that are desirable to treat with the microemulsions of the present disclosure are diverse, including but not limited to living and non-living surfaces, surfaces to be cleaned and/or protected, agricultural materials, surfaces associated with domiciles, institutions, or other buildings, and roadway or bridge surfaces.

In some embodiments, re-solubilization of the microemulsion system: (i) removes the deposited oil/surfactant system, and/or (ii) removes other materials solubilized by the oil/surfactant system, and/or (iii) removes only part of the oil/surfactant system originally deposited, or (iv) exhibits combinations of these behaviors. Because of these features, the types of surfaces that are desirable to treat with the microemulsions of the present disclosure are diverse, including but not limited to living and non-living surfaces, surfaces to be cleaned and/or protected, agricultural materials, surfaces associated with domiciles, institutions, or other buildings, and roadway or bridge surfaces.

In some embodiments, a cycle of self-dilution and drying through concentration may be repeated without altering the microemulsion. For example, if the microemulsions of the present system are dried on, for example, a cotton fabric surface and then re-formed by application of hot water, the fabric does not show evidence of oil deposition. Remarkably, if this process is performed on a triglyceride stain on cotton fabric, for example, olive oil, the stain is also removed or substantially reduced upon dissolution. A comparable oil stain is permanent to treatment with an alkylpolyglucoside for example, and even to an aqueous solution of a suitable microemulsion-forming surfactant of the sort used in the present disclosure, even if the solution is substantially dried to permit largely anhydrous interaction between the surfactant and fiber-embedded oil.

In some embodiments, solutions of the surfactant alone may be dried and readily reconstituted. This property of reconstitution may be usefully applied to facilitate dispensing of appropriately small quantities of a surfactant or a microemulsion to a surface as a dilute solution, that will dry to deposit an appropriate quantity of the surfactant or microemulsion product. For example, a fabric stain might require only milligram quantities of surfactant.

In some embodiments, the dilute microemulsion may be applied to a surface and rinsed away without drying, nonetheless effectively solubilizing a stain or soil mark. Without being bound by theory, it is understood that the microemulsion constitutes a liquid of polarity intermediate to its component phases alone, and with solubilizing qualities that may therefore substantially differ from the constituent ingredients. Thus it is understood that the microemulsion may act as a solvent in its own right, which may explain the particular effectiveness of the combined systems described herein in removing oil, food, and other soils that are intractable when treated by the components used singly.

### Drying Behavior in the Presence of Polymers

In some embodiments, when the microemulsion systems of the present disclosure dry by evaporation of the water continuous phase, polymers dissolved to varying degrees in the oil and/or water phases will be concentrated with respect to the aqueous fraction. Concentration of polymeric species can increase their tendency to interact, and counter-ions can promote the formation of salt linkages which may be poorly reversible. Thus, without being bound by theory, it is believed that as the products of the present disclosure dry, selective precipitation and solidification of dissolved polymeric species may occur, creating a porous matrix in which the microscopic oil domains remain entrapped. This resulting matrix may be poorly soluble in water or simple water and surfactant systems, yet readily degradable under oxidizing or strongly ionic conditions.

In some embodiments, the dilute microemulsions of the present disclosure comprising only oil and surfactant produce an oily film once dried that may be resuspended in water. If a composition further comprises systems of polymeric species, upon drying a precipitated polymer matrix may be observed under microscopic inspection. A system comprising 1% P9, 0.1% CTG, and 0.01% argan oil and further comprising 0.1% chitosan (chitosan CsG, Earth Supplied Products, Naples, Fla.) forms a continuously fine-grained film when dried. A system with the same composition that further includes 0.1% sodium alginate (Manugel GSB, FMC Health & Nutrition, Philadelphia, Pa.) deposits microscopic aggregates of continuously varied size and density, showing a radially striate structure surrounding the center of the dried droplet. It appears that the charged polymers undergo a concentration-dependent association, forming more and more extended linkages as the polymer concentration increases in the drying droplet.

Additional Components of the Compositions and/or Microemulsions

In some aspects, the compositions and/or microemulsions of the present disclosure may further modified to comprise additional useful properties or activities, including but not limited to: fragrance, flavor, repellency to insects, color, pharmacologic activity, and UV-absorbing properties, and/or support for solution or dispersion of secondary materials that can be delivered by means of the dispersions described herein. In some embodiments, materials that can be usefully incorporated directly into the compositions and/or microemulsions may be oils themselves or may be oil-soluble, forming an oil solution that can be incorporated in the present disclosure, or they may be aqueous soluble. If the compositions and/or microemulsions of the present disclosure are subsequently diluted, the materials used for dilution may similarly possess any of a wide variety of useful properties, and additionally may contain surfactants or solvents that contribute additional cleaning or other beneficial capacities to a formulation, while not being required to form the primary composition and/or microemulsion itself.

Some materials that may be beneficially included in the present disclosure may be more soluble in such compositions and/or microemulsions than in either the aqueous or the oil phases alone, due to the extensive area of the uniquely polarized phase interface presented by compositions and/or microemulsions systems.

In some embodiments, useful photo-absorbers include but are not limited to: p-aminobenzoic acid (PABA), avobenzone, 3-benzylidine camphor, bismidazylate, diethylamino hydroxybenzoyl hexyl benzoate, diethylhexyl butamido triazone, dimethicodiethylbenzal malonate, ecamsule, ensulizole, homosalate, isoamyl p-methoxycinnamate, 4-methylbenzylidine camphor, octocrylene, octyl dimethyl PABA, octylmethoxycinnamate (hereafter OMC), octyl salicylate, octyl triazone, bis-ethylhexyloxyphenol methoxyphenyl triazine, methylene bis-benzotriazolyl tetramethylbutylphenol, oxybenzone, PEG-25 PABA, polyacrylamidomethyl benzylidine camphor, and sulisobenzone. For example, 1 part octylmethoxycinnamate to 4 parts P9 readily forms a microemulsion of the form of the present disclosure when diluted in water.

In some embodiments, compositions and/or microemulsions of the present disclosure comprise zinc oxide and/or titanium oxide. The compositions and/or microemulsions of the present disclosure are not destabilized by the presence of inorganic pigments or sunscreen agents including oxides of zinc or titanium, or coated or functionalized forms of these materials, and these compositions and/or microemulsions can therefore be used in conjunction with such inorganic absorbers co-dispersed in a continuous phase, realizing the benefits of both ingredients.

As a non-limiting, illustrative example, the photo-absorber octinoxate (GalSORB OMC (HP), Tri-K Industries, Denville, N.J.) can be combined with D9 surfactant in a ratio of at least 5 parts surfactant to octinoxate, and in any ratio with water to form the compositions of the present disclosure. Higher ratios of surfactant to octinoxate do not impact clarity of the product. Similarly, a blend of the UV absorbers octocrylene (Escalol 597, Ashland Chemical, Covington, Ky.) and avobenzone GalSORB Avobenzone, Tri-K Industries) at a ratio of 24 parts avobenzone and 64 parts octocrylene can be combined with D9 surfactant in a ratio of at least 6 parts surfactant to dimethysiloxane, and in any ratio with water to form the compositions of the present disclosure. Higher ratios of surfactant to the sunscreen blend do not impact clarity of the product.

In some embodiments, water soluble or oil-soluble vitamins, nutritional supplements, and/or derivatives thereof may be usefully incorporated into the compositions and/or microemulsions of the present disclosure, including but not limited to: 2-methyl-1,4-naphthoquinone (3-) derivatives, cholocalciferol, tocopherol, retinol, esterified ascorbates, fat-soluble forms of thiamin, riboflavin, niacin, pantothenic acid, pyridoxine, biotin, folates, and cyanocobolamins, carotinoids, coQ10, curcumin, omega-3-fatty acids. For example, a composition and/or microemulsion of the present disclosure can be formed by combining 2 parts of a solution of Vitamin D3 in corn oil (100,000 IU/g Vitacyclix, Tuckahoe, N.Y.) with 8 parts CTG and 90 parts P9, followed by water dilution.

In another non-limiting embodiment of the present disclosure, a microemulsion is readily formed in water by dilution of a solution of 1 part of the insect repellent ethyl butylacetylaminopropionate (IR3535, Merck & Co., Kenilworth, N.J.) and 9 parts P9. The invention may comprise any known repellent, or combinations thereof, including without limitation, benzaldehyde, N,N-diethyl-m-toluamide (DEET), dimethyl carbate, dimethyl phthalate, hydroxyethyl isobutyl piperidine carboxylate (Icaridin), indalone, metofluthrin, permethrin, tricyclodecenyl allyl ether, birch (Betula sp) bark, bog myrtle (Myrica gale), catnip extracts, citronella oil, citrus oils, limonene, lemon eucalyptus (Corymbia citriodora) oil, neem oil, lemongrass oil, and tea tree oil. As a non-limiting example, 1 part N,N-diethyl-m-toluamide (DEET) may be dissolved in 9 parts P9, and the mixture thus formed can be diluted to any degree by adding water without loss of visual clarity. In another embodiment, substitution of, for example, ethyl butylacetylaminopropionate, or limonene in place of DEET produces a product of similar clarity. In another embodiment of the invention, a composition of 20 g IR3535, 20 g PEG-6 caprylic/capric triglycerides, and 60 g water forms a clear microemulsion.

In some embodiments, other materials may be usefully dissolved in the oil or aqueous phases of the present disclosure, or dissolved after formation of the microemulsion. For example, hydroxystearic acid (CASID HAS, Vertellus Performance Materials, Greensboro, N.C.) and polyamide-3 (OLEOCRAFT MP-30, Croda, Mill Hall, Pa.) are both effective gelling additives for some intermediate polarity oils, and can for instance, create a non-flowable gel when dissolved in OMC at 1-5% use level. The resulting gels are readily incorporated in the systems of the present disclosure. Interestingly, while HSA is very poorly solvated by water, it is readily dissolved with heating at 5% relative to OMC content in a microemulsion of OMC formed as taught herein, and cools to form a water clear product.

It will be obvious to one of ordinary skill in formulation of products for personal care or home care that a variety other materials may be usefully dispersed in or combined with the systems of the present disclosure, including preservatives, fragrances, colorants, surfactants, abrasives or exfoliating materials, or other beneficial or active agents that provide additional functionality.

### Optical Transparency

The compositions and/or microemulsions of the present disclosure are readily distinguished from comparable compositions and/or microemulsions or dispersions by their optical transparency. The compositions and/or microemulsions of the present disclosure can be formed so as to be optically transparent. Optical transparency (or clarity) of a composition and/or microemulsion can be determined by any method known in the art. For example, optical transparency can be determined through use of a turbidimeter, an instrument for measuring the turbidity of a liquid suspension. In some embodiments, the optically transparent compositions and/or microemulsions of the present disclosure have a turbidity ranging from 0-15 Nephelometric Turbidity Units (NTUs), from 0-10 NTUs, from 0-8 NTUs, from 0-6 NTUs, from 0-5 NTUs, from 0-4 NTUs, or from 0-2 NTUs. In a particular embodiment, the optically transparent compositions and/or microemulsions of the present disclosure have a turbidity of 10 NTUs or less.

In some embodiments, the compositions and/or microemulsions remains optically transparent upon dilution. In some embodiments, the compositions and/or microemulsions remains optically transparent following dilution that ranges from a two-fold to 10-fold dilution, a 5 fold to 50 fold dilution, a 10 fold to 100 fold dilution, a 50 fold to 500 fold dilution, a 100 fold to 1000 fold dilution, or a 100 fold to 10,000 fold dilution with aqueous phase or oil phase.

### III. Methods of Using Compositions and/or Microemulsions

Home Care Compositions and Methods of their Use

In some aspects, provided herein are home care compositions comprising, consisting of, or consisting essentially of the compositions and/or microemulsions disclosed herein. In some embodiments, the charged polymer complex encapsulates the dispersion phase. In some embodiments, the charged polymer complex forms an interconnected network of fibers.

In some aspects, provided herein are home care compositions comprising, consisting of, or consisting essentially of compositions and/or microemulsions disclosed herein. In some embodiments, the polymer comprises, consists of, or consists essentially of: one or more amphiphilic polymers, one or more amphiphilic charged species, and/or combinations thereof.

In some aspects, provided herein are methods of treating a surface with a home care composition, the method comprising, consisting of, or consisting essentially of applying the home care composition comprising the composition and/or microemulsion disclosed herein of the present disclosure to the surface, thereby treating the surface. In some embodiments, the surface is a textile. In some embodiments, the surface is a kitchen surface. In some embodiments, the kitchen surface is selected from the group consisting of a floor, a countertop, a stovetop, a sink, and an appliance. In other embodiments, the surface is leather, vinyl, or a synthetic leather material. In some embodiments, the surface is a wall, floor, or ceiling. In other embodiments, the surface comprises a plant.

In some embodiments, treatment of the surface results in alleviating, abating, or ameliorating one or more conditions of the surface. In other embodiments, treatment results in preventing the development of one or more conditions. Non-limiting examples of conditions of the surface include one or more of the following: damage, soil, scratches, pitting, erosion, chemical damage, radiation-induced damage, wear, thinning, non-planarity, visual non-uniformity, warping, staining, hydrophobicity, hydrophilicity, dryness, loss of color, streaks, mold and bacterial growth, formation of a biofilm, susceptibility to infestation by insects, and weakness. In yet other embodiments, treatment results in imparting a desired property to the surface. Non-limiting examples of a desired property include adding moisture, shine, cleanliness, lubrication, strength, planarity, smoothness, slip, gloss, hydrophobicity or hydrophilicity, wetting contact angle, visual uniformity, chemical resistance, traction, hardness, resilience, flexibility, protection from ultraviolet light, colorfastness, resistance to accumulation of soil, resistance to redeposition of soil during cleaning, abatement of or resistance to mold and/or bacterial growth and/or biofilm formation, abatement of or resistance to infestation, and resistance to damage.

In some aspects, provided herein are methods of laundry care, the method comprising, consisting of, or consisting essentially of applying the home care composition comprising the composition and/or microemulsion of the present disclosure to the laundry, thereby treating the laundry. As used herein, “laundry” refers to clothes and/or linens that need to be washed. In some embodiments, treatment comprises washing the laundry. In some embodiments, treatment comprises removal of one or more stains from the laundry. In some embodiments, the method further comprises prevention of soil-redeposition on the laundry. In some embodiments, the method further comprises removal of odors.

### Cleansing Home Care Compositions Comprising Microemulsions

In some aspects, the home care compositions of the present disclosure can be diluted with solutions of a variety of surfactants to produce cleansing compositions that also may deliver the dispersed oil phase into contact with a treated surface, acting as a benefit agent in the cleansing composition. Further, the presence of selected charged polymeric species in the microemulsions can promote or inhibit deposition of the dispersed oil phase. The microemulsions of the present disclosure are useful in removing soils from textiles, and can help to prevent re-deposition of soils once they have been removed. Treatment of surfaces with the microemulsions of the present invention can disrupt microbial and other biofilms, aiding in removal of such biofilms, and can inhibit post-treatment formation of biofilms. Importantly, cleansing surfactants in such compositions are not required nor specifically facilitative of microemulsion formation, and generally such surfactants are not suitable for forming the microemulsions of the present disclosure. Rather these additional surfactants form the cleansing system to which the microemulsion may be added, already fully formed, as a benefit agent. Suitable anionic, non-ionic, amphoteric and/or zwitterionic, and/or cationic surfactant solutions, as non-limiting examples of each type of surfactant, disodium laureth sulfosuccinate (Mackanate ELK, Solvay USA, Princeton, N.J.), Coco-glucosides (PUREACT GLUCO C, Innospec Performance Chemicals, Salisbury, N.C.) or Cetrimonium Chloride (Jeequat CT-29, Jeen Intl. Corp., Fairfield, N.J.). However, diverse surfactants of each of these types are known, and essentially any of these may be combined with the microemulsions taught herein.

Examples of suitable anionic surfactants include but are not limited to alkyl sulfates, alkyl ether sulfates, alkaryl sulfonates, alkyl succinates, alkyl sulfosuccinates, N-alkoyl sarcosinates, alkyl phosphates, alkyl ether phosphates, alkyl ether carboxylates, alkylamino acids, alkyl peptides, alkoyl taurates, carboxylic acids, acyl and alkyl glutamates, alkyl isethionates, and alpha-olefin sulfonates, fatty acid soaps, and water-soluble salts thereof.

Representative suitable nonionic surfactants include but are not limited to aliphatic primary or secondary linear or branched chain acids, alcohols or phenols, alkyl ethoxylates, alkyl phenol alkoxylates (especially ethoxylates and mixed ethoxy/propoxy), block alkylene oxide condensate of alkyl phenols, alkylene oxide condensates of alkanols, ethylene oxide/propylene oxide block copolymers, semi-polar nonionics (e.g., amine oxides and phospine oxides), alkyl amine oxides, mono or di alkyl alkanolamides and alkyl polysaccharides, sorbitan fatty acid esters, polyoxyethylene sorbitan fatty acid esters, polyoxyethylene sorbitol esters, polyoxyethylene acids, and polyoxyethylene alcohols.

In addition, amphoteric and zwitterionic surfactants suitable for combination with the microemulsions herein comprise: alkyl betaines, alkyl amidopropyl betaines, alkyl sulphobetaines, alkyl glycinates, alkyl carboxyglycinates, alkyl amphopropionates, alkyl amidopropyl hydroxysultaines, acyl taurates and acyl glutamates.

Suitable cationic surfactants include but are not limited to alkyl amines, alkyl imidazolines, ethoxylated amines, quaternary compounds, and quaternized esters.

Other suitable surfactants are described in McCutcheon's Emulsifiers and Detergents (North American and International Editions, by Schwartz, Perry and Berch) and a variety of other surfactant references commonly known to surfactant formulators.

While amounts of surfactant can vary widely, amounts which are often utilized generally range from about 0.5% to about 80%, or from about 5% to about 60%, and preferably from about 6% to about 30% or most preferably from about 8% to 20% weight based upon the total weight of the composition.

### Personal Care Compositions

In some aspects, provided herein are personal care compositions comprising, consisting of, or consisting essentially of the composition and/or microemulsion disclosed herein. In some embodiments, the charged polymer complex encapsulates the dispersion phase. In some embodiments, the charged polymer complex forms an interconnected network of fibers.

In some aspects, provided herein are personal care compositions comprising, consisting of, or consisting essentially of the composition and/or microemulsion disclosed herein. In some embodiments, the polymer comprises, consists of, or consists essentially of: one or more amphiphilic polymers, one or more amphiphilic charged species, and/or combinations thereof.

In some embodiments, the personal care composition is a hair care composition. In some embodiments, the hair care composition is selected from the group consisting of: shampoo, conditioner, treatment, mask, styling agent or color protecting treatments and technologies. In some embodiments, the hair care composition softens the hair. In some embodiments, the personal care composition is a skin care composition. In some embodiments, the skin care composition is a moisturizer, cream, lotion, or body oil.

In some embodiments, the oil phase of the personal care composition comprises one or more of the group consisting of: coconut oil, argan oil, citrus oil, CTG, and octyl methoxycinnamate. In some embodiments, the surfactant is P9 or SODC.

In some embodiments, personal care composition further comprise proteins, products of protein hydrolysis, peptides, amino acids, nucleic acids, oligomers, plasmids, sense- and anti-sense ribonucleic acid and deoxyribonucleic acid sequences and conjugates of proteins and nucleic acids may all be usefully included as additives to the composition and/or microemulsions of the present disclosure. Such materials may, as non-limiting examples, confer useful properties such as promotion or inhibition of biological processes, induction or regulation of protein expression, enhancement of material properties such as detectability or counterfeit-deterrence, delivery of materials into cells or tissues, or enhancement or inhibition of deposition onto surfaces such as skin, hair, mucosa, teeth, or nails.

In some embodiments, the personal care composition further comprises at least one peptide. In some embodiments, the peptide is a pentapeptide comprising amino acids selected from the group consisting of: cysteine, arginine, proline, and serine. In some embodiments, the pentapeptide has an amino acid sequence consisting of CCRPS (SEQ ID NO: 1).

In some embodiments, the personal care compositions can be usefully incorporated into cleansing compositions for skin. For example, a composition comprising 0.1 g sodium alginate, 0.01 g polyquaternium 10, 0.1 g Argan oil, 1.0 g CTG, 10.0 g P9 and 30.0 g water can be added to a hot bath as a skin moisturizer, providing excellent skin feel and lubricity. Incorporation of an additional 1 g coco glucoside (PureAct Gluco C, Innospec Inc, Littleton, Colo.) and 1 g cocamidopropyl betaine (Rita Corp, Crystal Lake, Ill.) into this composition produces a luxurious foaming bath composition that retains the beneficial skin feel of the microemulsion composition.

In some aspects, provided herein are methods of treating hair, the methods comprising, consisting of, or consisting essentially of applying a personal care composition to the hair, thereby treating the hair. In some embodiments, the hair is artificially colored. In some embodiments, the treatment prevents and/or reduces washout of the artificial color. In some embodiments, the treatment comprises repair or prevention of hair damage. In some embodiments, the hair damage comprises split-ends.

In some embodiments, the personal care composition is applied to the hair for about 30 seconds to about 2 minutes, about 30 seconds to about 3 minutes, about 30 seconds to about 5 minutes, about 30 seconds to about 90 seconds, about 60 seconds to about 120 seconds, about 60 seconds to about 3 minutes, about 60 seconds to about 5 minutes, or about 60 seconds to about 10 minutes. In some embodiments, the personal care composition is washed out of the hair following application.

In some embodiments, the treatment comprises deposition of a photo-absorber, thereby protecting the hair from sun damage.

In some aspects, provided herein are methods of treating an internal or external surface of a subject, the methods comprising, consisting of, or consisting essentially of applying a personal care composition to the internal or external surface of the subject, thereby treating the internal or external surface of the subject. In some embodiments, the internal surface is one or more of the group selected from: teeth, oral cavity, and mucosal surface. In some embodiments, the external surface is one or more of the group selected from: skin, nail, and scalp. In some embodiments, the external surface is a nail comprising a fingernail or a toenail. In some embodiments, the external surface is skin. In some embodiments, the external surface is hair. In some embodiments, the treatment comprises deposition of a photo-absorber, thereby protecting the surface from sun damage.

In some embodiments, treatment alleviates, abates, or ameliorates one or more conditions experienced by the subject. In some embodiments, treatment prophylactically prevents the development of one or more conditions on the subject, Non-limiting examples of conditions include one or more of the following: hair damage, split ends, dry hair, dry skin, pruritis, eczema, aging, sun damage, chemical damage, loss of artificial hair color, loss of moisture, oil build up, vitamin deficiency, finger- or toenail fragility, nail dullness, thickening or cracking of nail cuticles, hair fragility or breakage, hair loss, formation of dental caries, halitosis, periodontal disease, skin discoloration, skin thickening, loss of skin flexibility, loss of skin elasticity, skin inflammation, acne or skin ulcers, wrinkles, and/or sagging of skin. In some embodiments, treatment imparts a desired property to the treated surface of the subject. Non-limiting examples of a desired property include adding moisture (e.g., via occlusive action, humefactant action, or through restoration of deficient materials), shine, protection from ultraviolet light, absorbing or reflecting ultraviolet light, resistance to accumulation of soil, resistance to damage, repelling insects, and wound healing, evening of skin tone, lightening of skin, darkening of skin, restoration of skin elasticity or flexibility, reduction of inflammation or acne or skin ulcers, tightening of skin, prevention of hair tangling, fragility or breakage, ease of hair combing, prevention of dental caries or periodontal disease or halitosis, improvement of finger- or toenail flexibility, smoothness, reflectivity, and/or reduction of nail cuticle thickening or cracking.

In some embodiments, the subject is a mammal. In particular embodiments, the subject is a human, equine, bovine, porcine, feline, canine, murine, rat, or non-human primate. In preferred embodiments, the subject is a human. The subject may or may not be in need of treatment with a composition of the present disclosure.

### Sunscreens

In some aspects, one or more sunscreens are incorporated into the compositions and/or microemulsions of the present disclosure. For example 4 g octylmethoxycinnamate, hereafter OMC, (GalSORB OMC, Galaxy Surfactants Ltd. Denville, N.J.), a common UVB absorber, will form a water-clear microemulsion when first dissolved in 16 g P9, and then diluted with 80 g of 50° C. water. The resulting system is continuously dilutable and readily combined with for example cleansing surfactant compositions or hair conditioner compositions. Similarly 1 g of the common UVA absorber Avobenzone (Butyl Methoxydibenzoylmethane, GalSORB Avobenzone, Galaxy Surfactants Ltd. Denville, N.J.) dissolved in 4 g Octocrylene (GalSORB Octocrylene, Galaxy Surfactants Ltd. Denville, N.J.) and 4 g of Finnsolve TN (C₁₂₋₁₅ Alkyl Benzoate, Innospec Inc., Englewood, Colo.) will form a water-clear microemulsion when combined with 40 g P9 and diluted with water.

In some embodiments, compositions and/or microemulsions of the present disclosure comprise zinc oxide and/or titanium oxide. The compositions and/or microemulsions of the present disclosure are not destabilized by the presence of inorganic pigments or sunscreen agents including oxides of zinc or titanium, or coated or functionalized forms of these materials, and these microemulsions can be therefore be used in conjunction with such inorganic absorbers co-dispersed in a continuous phase, realizing the benefits of both ingredients.

Haircare Compositions and/or Microemulsions

In one aspect, the present disclosure provides compositions and/or microemulsions as disclosed herein for personal care comprising very small dispersed domains of oil that may bind amphiphilic polymers. Being of sub-micron dimensions, such oil droplets may access fine topologies, for example micro-fractures, pores, or other voids formed in damaged hair, or may enter pores or follicles more efficiently than larger droplets. Being superficially surfactant-rich such droplets may also more efficiently interact with surface-available lipids on hair, skin, and component structures thereof. Further, upon accessing these smaller void spaces, charged polymers associated with these droplet structures may further interact to form complex lattice structures at an ultrafine scale, thereby producing repair of hair at a previously unattainable level.

While the exact mechanisms by which repair of hair damage occurs associated with charged polymer interaction and dispersed oil droplets are not fully understood or described, several observations support the idea that this capacity to repair damage is altered in the present composition and/or microemulsion systems relative to the larger droplet sizes described in prior art.

The compositions and/or microemulsions of the present disclosure can be usefully added to existing hair care formulations, including without limitation, shampoos, conditioners, leave-in treatments, styling aids, and products that modify hair, such as relaxers, straighteners, softeners, moisturizers, bleaches, and colorants. For example, a microemulsion comprising 1% CTG and 10% P9 was mixed with the color solution and the developer solution of a commercial hair coloring kit (Colorsilk, Revlon Inc., New York, N.Y.), each with no change in clarity, precipitation, or other apparent incompatibility. When combined, the interaction of peroxide, base, and color components of the kit (Medium Auburn 42) developed an intense color indistinguishable from the combination in the absence of the microemulsion.

### Color-Retention in Artificially Colored Hair

In specific testing of shampoo formulations on artificially colored hair, suspensions of larger droplets encapsulated in alginate/chitosan walled microcapsules described in prior art (commercially available as Vegabead Argan products, ESP) provided limited reduction of color washout in dyed hair samples. However, replacement of the Vegabead material with an identical use level of one of the microcapsule suspensions of the present disclosure and no other formulation change produced a dramatic, readily visible reduction in color washout after a single use, with roughly 10-fold reduction of color in the collected rinse water, very strongly outperforming all other products tested as well as a water-only control.

Without being bound to a particular interpretation, it is possible that the microemulsions of the present disclosure in conjunction with charged polymer content in the product provided this color-retention benefit through a mechanism that might include: 1. effective penetration into surface micro-topological voids wherein artificial hair color is normally deposited; 2. association of charged polymers with ionized sites in chemically damaged hair; 3. concentration and concomitant formation of matrices of linked charged polymers; and 4. non-washout of the deposited matrices and reduced water exchange into the voids, thus retaining color.

Split-End Repair with Single Charged Polymers

Effective semi-permanent repair of split-end hair damage has been shown in prior art using charged polymer complexes and more recently using microcapsules formed from similar complexes of charged polymers. Charged or uncharged polymers alone do not generally produce repair of split ends in the authors experience and prior art does not support that general concept. In fact, many cationic polymers can enhance the visibility and geometric angle of split end and other damage, contrary to their other beneficial effects on hair. Therefore the authors were surprised when what was nominally an experimental negative control, a dilute CTG microemulsion further comprising 0.01% chitosan (ESP) but no alginate or other polymeric counter-ion produced repair of split-end damage on contact. This system further produced very robust repair in a simple model hair product comprising the microemulsion with chitosan and further comprising 2% cetrimonium chloride (CTK) (Incroquat CTC-30, Croda). A comparable system of chitosan and CTK did not produce repair, and nor did a system of microemulsion and CTK alone. The repair by the microemulsion and a charged polymer alone is unanticipated and not readily explained by current understanding of the repair mechanism.

Without being bound to a particular interpretation, while associative interaction between the charged polymer and charged sites on the damaged hair surface is well described, that association does not ordinarily produce repair activity. It is possible that the concentration of amphiphilic polymer in the interstitial spaces between microscopic oil domains during product drying led to formation of poorly soluble complexes or sufficient wetting of the polymer by the oil phase to inhibit re-solubilization, and thus provided a durable semi-permanent repair. Generally, polymeric Lewis acids comprising those described in U.S. 2008/0138420A1, Microencapsulation product and process may be usefully incorporated in compositions comprising the microemulsions taught in the present disclosure.

Non-limiting examples of polymers useful in combination the present disclosure comprise: acacia gums, agar, polyacrylic acid, albumins, carbomers, cassia gum, cellulose gums, chitosan, chondroitin, curdlan, gelatin, dextran, fibrin, fulcelleran, gellan gum, ghatti gum, guar gum, gum tragacanth, heparin, hyaluronic acid, karaya gum, locust bean gum, pea protein, pectin, polyoxyethylene-polyoxypropylene and other synthetic block copolymers, pullulan, starch, tara gum, whey protein, xanthan gum, and zein, and ions and salts of these materials. Generally, polymeric materials of molecular weight above 1000 amu presenting at least two carboxylic acid reactive groups, or combinations of these materials, are suitable Lewis acid components.

In addition to the Lewis acids listed herein, Lewis base ions and their water-soluble salts comprising those described in U.S. 2008/0138420A1 may also be usefully included in compositions comprising the microemulsions of the present disclosure. Examples of useful Lewis base ions comprise: benzalkonium, cetylpyridinium, chitosan, cocodimonium hydroxypropyl hydrolyzed keratin, cocoglucosides hydroxypropyl, hydroxypropyltrimonium hydrolyzed wheat protein, hydroxypropyl oxidized starch PG-trimonium, PEG-3 dioleylamidoethylmonium methosulfate, laurdimoniumhydroxypropyl decylglucosides, polyquaternium-10, polyquaternium-11, polyquaternium-78, polyquaternium-80, polyquaternium-81, polyquaternium-88, polyquaternium-101, quaternium-79 hydrolyzed silk protein, silicone quaternium-17, silicone quaternium-8, starch hydroxypropyltrimonium, steardimonium hydroxyethylcellulose, steardimonium hydroxypropyl panthenyl PEG-7 dimethicone, cocodimonium hydroxyethylcellulose, polyvinylamine and generally, any water-soluble quaternary amine or similar Lewis base.

### IV. Kits

In some aspects, also provided herein are kits comprising, consisting of, or consisting essentially of the compositions and/or microemulsions disclosed herein, and instructions for use. In some embodiments, the charged polymer complex comprises, consists of, or consists essentially of: one or more amphiphilic anionic charged polymers, one or more amphiphilic cationic charged species, and/or combinations thereof. In some embodiments, the charged polymer complex microemulsion is a home care composition or a personal care composition. In some embodiments, kits further comprise components that can be combined to form such compositions and/or microemulsions. The compositions may also be part of a formulation comprising additional materials

In some aspects, also provided herein are kits comprising, consisting of, or consisting essentially of dilutable compositions and/or microemulsions disclosed herein, and further comprising instructions for use. In some embodiments, kits further comprise components that can be combined to form such compositions and/or microemulsions. The compositions may also be part of a formulation comprising additional materials.

### V. Examples

The following examples are given to illustrate the present disclosure. It should be understood, however, that the invention is not to be limited to the specific conditions or details described in the examples.

### Example 1. Capric/caprylic Triglycerides Microemulsion

A 10 g aliquot of capric/caprylic triglycerides (Lexol GT, Inolex Chemical Co., Philadelphia, Pa., hereafter CTG) was combined with 80 g of a 62% solution of SODC and warmed to 75° C. to facilitate mixing. The materials were stirred until no mixing striations were observed. To this mixture, 110 g of deionized (DI) water were added at 75° C., and stirred until a clear dispersion was formed. The material thus formed was stable to freezing and thawing, and in storage at 45 C for at least one month. This product was entirely water-dilutable and self-dispersing.

### Example 2A. CTG Macroemulsion

A 10 g aliquot of CTG was combined with 0.1 g of sorbitan monooleate (Jeechem SMO, Jeen Corp, Fairfield N.J.) and 0.1 g polysorbate 20 (Ritabate 20, Rita Corp, Crystal Lake, Ill.) in 89.8 g DI water, warmed to 60° C. to facilitate mixing. The materials were homogenized to form a fine, opaque, white emulsion. The resulting material showed creaming instability within 1 hr, forming a dense white oil-rich layer at the surface. Within one week at 45° C., pooled oil was visible at the surface of the product. While the product was water-dilutable, creaming instability remained a problematic characteristic of this macroemulsion, distinguishing it from the correctly formed microemulsions of the present disclosure.

### Example 2B: CTG Macroemulsion for Comparison to Microemulsion

A 10 g aliquot of CTG was combined with 80 g of polysorbate 20 (Ritabate 20, Rita Corp, Crystal Lake, Ill.) and warmed to 75° C. to facilitate mixing. To this mixture, 110 g of deionized (DI) water were added at 75° C. When stirred and cooled as in Example 1, the product formed an opaque emulsion. This product was further processed by application of very high shear using a rotor-stator homogenizer at 5000 rpm for 5 minutes, with no improvement in clarity, indicating that droplet sizes remained large enough to demonstrate strong wavelength-independent (Mie) scattering, visible evidence that the droplets produced in Example 2 were very substantially larger than those produced in Example 1. The resulting material showed creaming instability within 1 hr, forming a dense white oil-rich layer at the surface. Within one week at 45° C., pooled oil was visible at the surface of the product. While the product was water-dilutable, creaming instability remained a problematic characteristic of this macroemulsion, distinguishing it from the correctly formed microemulsions of the present disclosure.

### Example 3. Lemon Oil Microemulsion

A 10 g aliquot of Lemon Oil (ESP) was combined with 60 g SODC and mixed by stirring. To this stirring solution, 240 g DI water was added at room temperature. The material thus formed was stable to freezing and thawing, and in storage at 45 C for at least one month. This product was entirely water-dilutable and self-dispersing. Further, in testing, various dilutions of the product in water (beyond 1:10) disrupted scent trails and actively repelled common Argentinian ants, preventing colonies from renewing established trails to food sources after cleaning floor, counter and wall surfaces with the dilution. In additional testing the product further effectively removed and dispersed mildew and algae from surfaces with several years accumulated growth.

### Example 4. CTG/Argan Microemulsion Including SODC

A 9 g aliquot of CTG was combined with 1 g of argan (Argania spinosa) seed oil (Earth Supplied Products LLC, Naples, Fla., hereafter ESP), and 80 g of a 62% solution of SODC and warmed to 75° C. to facilitate mixing. The materials were stirred until no mixing striations are observed. To this mixture, 110 g of deionized (DI) water were added at 75° C., and stirred until a transparent dispersion is formed. The material thus formed was stable to freezing and thawing, and in storage at 45 C for at least one month. This product was entirely water-dilutable and self-dispersing.

### Example 5. CTG/Argan Microemulsion Including P9

A 9 g aliquot of CTG was combined with 1 g of argan (Argania spinosa) seed oil (Earth Supplied Products LLC, Naples, Fla., hereafter ESP), and 80 g of P9 and warmed to 60° C. to facilitate mixing. The materials were stirred until no mixing striations are observed. To this mixture, 110 g of deionized (DI) water were added at 60° C., and stirred until a transparent dispersion was formed. The material thus formed was stable to freezing and thawing, and in storage at 45 C for at least one month. This product was entirely water-dilutable and self-dispersing.

### Example 6. OMC Microemulsion

A 4 g aliquot of OMC was combined with 16 g P9, and then diluted with 80 g of 50° C. water. The resulting product was a water-clear microemulsion similar to others described herein.

### Example 7. OMC Microemulsion

A 1 g aliquot of Avobenzone was dissolved in 4 g Octocrylene and 5 g of octylsalicylate, and then combined with 60 g P9. The clear product was then diluted with 230 g of 50° C. water. The resulting product was a water-clear microemulsion similar to others described herein.

In some embodiments, the present disclosure may further comprise multiple benefit agents with different functions, such as a hair benefit oil combined with a UV absorber.

### Example 8. OMC/Argan Microemulsion

A 10 g aliquot of OMC was combined with 1 gram of argan oil, and this mixture was then combined with 89 g P9. The resulting clear product was then diluted with 300 g of 50° C. water. The resulting product was a water-clear microemulsion similar to others described herein.

In another aspect, the invention provides microcapsules formed from the microemulsions described herein.

### Example 9. Alginate/Chitosan Microencapsulated Microemulsion

To 90 g of P9 was added 9 g of capric/caprylic triglycerides and 1 g argan oil, and the mixture warmed to 60° C. and stirred until a clear product was obtained. To this product was added 395 g of water and 5 g of a 0.01% solution of sodium alginate. The mixture was warmed to 60° C. and mixed until a clear product was again obtained. To this mixture finally was added 100 g of a 0.001% solution of chitosan. The material thus formed was stable to freezing and thawing, and in storage at 45 C for at least one month, remaining water-clear. This product was entirely water-dilutable and self-dispersing.

### Example 10. Microencapsulated Microemulsion Further Comprising a Peptide in the Charged Polymer Complex

To 90 g of P9 was added 9 g of capric/caprylic triglycerides and 1 g argan oil, and the mixture warmed to 60° C. and stirred until a clear product was obtained. To this product was added 394 g of water and 5 g of a 0.01% solution of sodium alginate, and 1 g of a 0.01% dilution of a pentapeptide, Cysteine-Cysteine-Arginine-Proline-Serine. This pentapeptide is a characteristic degradation product of Keratin Associated Protein-5 (KAP-5) which is highly expressed in human hair. Further, the forms a precipitate when mixed with chitosan solution, characteristic of materials suitable for inclusion in the charged complexes which are understood to contribute to hair damage repair in the present disclosure. The mixture was warmed to 60° C. and mixed until a clear product was again obtained. To this mixture finally was added 100 g of a 0.001% solution of chitosan. The material thus formed was stable to freezing and thawing, and in storage at 45 C for at least one month, remaining water-clear. This product was entirely water-dilutable and self-dispersing.

### Example 11. SODC/Chitosan Microencapsulated Microemulsion

To 25 g of the product of example 1, an aliquot of 2.5 g of a 0.01% solution of chitosan was added. Surprisingly, the product appearance changed from water-clear to hazy, showing wavelength-dependent light scattering. Further experimentation showed that the SODC is reactive with chitosan, forming a precipitate when solutions are combined. The product formed appeared to be an encapsulated microemulsion wherein the wall comprised the complex formed by chitosan and the surfactant material. This phenomenon was further surprising as neither the surfactant nor its component species appear to be anionic in nature, and an Lewis acid-Lewis base type precipitation of chitosan was therefore unexpected. The inventors initially suspected that precipitation might be related to solution pH, as chitosan is poorly soluble at high pH. However, reducing solution pH to 4 did not change precipitation behavior indicating some other, as yet unknown effect is at work. Despite the unknown cause, the complexation occurs, forming a new product.

### Example 12. Alginate/SODC/Chitosan Microencapsulated Microemulsion

An aliquot of 40 g of a 0.0025% solution of sodium alginate (Manugel GHB, FMC Health & Nutrition, Philadelphia, Pa.) was added to 50 g of the microemulsion of example 3. To the resulting mixture was added an additional 10 g of a 0.01% solution of chitosan (Chitosan HD, ESP). The material thus formed was stable to freezing and thawing, and in storage at 45 C for at least two months. This product was entirely water-clear, water-dilutable and self-dispersing. Initially it was believed that this product comprised simply an alginate/chitosan microcapsule, but in light of the findings of example 6, it is believed that both alginate and SODC are incorporated into the encapsulating wall.

### Example 13. Microemulsion with Cationic Polymer

To confirm that the chitosan precipitation observed in example 12 was simply due to the physical structure of the microemulsion, a 2.5 g aliquot of 0.1% chitosan solution was added to the microemulsion of example 4, which was formed using the C12-13 pareth-9 surfactant. No haziness was observed, and no reactivity between this surfactant and chitosan was observed in other experiments. The material thus formed was stable to freezing and thawing, and in storage at 45 C for at least one month, remaining water-clear. This product was entirely water-dilutable and self-dispersing.

In another aspect the invention provides products for hair damage treatment and a method of treating hair.

### Example 14. Split-End Repair by Alginate/Chitosan Microencapsulated Microemulsion

The water-clear product of example 9 was observed to accomplish semi-permanent repair of split-end hair damage after momentary immersion and brief air drying (60 s). Flexion of the repaired tip to a 45° angle did not dislocate nor rupture the repairs. Repair durability in simulated washing was further tested by repeated cycles of immersion in a 10% solution of Plantapon 611L (sodium laureth sulfate, lauryl glucoside and cocamidopropyl betaine, BASF Corp, Florham Park, N.J.), followed by water rinse and blow drying. Tip flexion after simulated washing did not produce failure of the repair in samples tested.

### Example 15. Split-End Repair by SODC/Chitosan Microencapsulated Microemulsion

The product of example 12 was observed to accomplish semi-permanent repair of split-end hair damage after momentary immersion and brief air drying (60 s). Flexion of the repaired tip to a 45° angle did not dislocate nor rupture the repairs. Repair durability in simulated washing was further tested by repeated cycles of immersion in a 10% solution of Plantapon 611L (sodium laureth sulfate, lauryl glucoside and cocamidopropyl betaine, BASF Corp, Florham Park, N.J.), followed by water rinse and blow drying. Tip flexion after simulated washing did not produce failure of the repair in samples tested.

### Example 16. Split-End Repair by Alginate/SODC/Chitosan Microencapsulated Microemulsion

The product of example 12 was observed to accomplish semi-permanent repair of split-end hair damage after momentary immersion and brief air drying (60 s). Flexion of the repaired tip to a 45° angle did not dislocate nor rupture the repairs. Repair durability in simulated washing was further tested by repeated cycles of immersion in a 10% solution of Plantapon 611L (sodium laureth sulfate, lauryl glucoside and cocamidopropyl betaine, BASF Corp, Florham Park, N.J.), followed by water rinse and blow drying. Tip flexion after simulated washing did not produce failure of the repair in samples tested.

### Example 17. Split-End Repair by Cationic Continuous Phase Microemulsion

For completeness, the product of example 13 was also evaluated for split-end repair performance, although previous data did not show chitosan alone to be effective in repairing split ends. Unexpectedly, it was also observed to accomplish semi-permanent repair of split-end hair damage after momentary immersion and brief air drying (60 s). Flexion of the repaired tip to a 45° angle did not dislocate nor rupture the repairs. Repair durability in simulated washing was further tested by repeated cycles of immersion in a 10% solution of Plantapon 611L (sodium laureth sulfate, lauryl glucoside and cocamidopropyl betaine, BASF Corp, Florham Park, N.J.), followed by water rinse and blow drying. Tip flexion after simulated washing did not produce failure of the repair in samples tested. Without being bound to a particular interpretation, it seems possible that the combination of the surfactant, oil and chitosan (which is understood to spontaneously bind to anionic sites on damaged hair) act cooperatively to wet the hair surface, permitting chitosan to act as a binding agent upon drying.

### Example 18. Enhancement of Encapsulated Microemulsion and Cationic Microemulsion Split-End Repair Activity by Quaternary Amines

The products of examples 9 through 13 were diluted to 1% levels in DI water containing 1% cetrimonium chloride. Split-end repair was produced by contact with any of these solutions. Split ends dipped in the 1% cetrimonium chloride solution alone did not produce repairs, although some samples momentarily wet closed by apparent surface tension effects.

### Example 19. Non-Repair of Split Ends by Microemulsion Alone

The products of examples 1-5 were duly tested for completeness, and when applied alone, each of them failed to accomplish semi-permanent repair of split-end hair damage after immersion and air drying.

In another aspect the invention provides products for hair-dye retention treatment and a method of treating hair.

### Example 20. Protection Against Wash-Out of Color-Treated Hair

The product of example 9 was incorporated in a shampoo and a conditioner formulation and these products were evaluated on color-treated hair to test for color wash-out. Virgin medium brown hair (level 5) was treated with Matrix Light Master Lightening Powder and Matrix 20 Volume Cream Developer, air dried, colored with Matrix 6RR+ and 20 Volume Cream Developer for 35 min, then air dried again (L'Oreal USA, Hudson Yards, N.Y.). As experimental controls, identical formulations without the product of example 5, as well as several commercial retail “color-protection” name brand shampoo and conditioner products and a water-only control were also tested. The formulations that included the product of example 5 showed dramatically less color wash-out, and reduction of color in rinse water samples was visibly obvious for the product set that included example 5, relative to all other samples, which showed stronger color washout in the rinse water.

Without being bound to a particular interpretation, it is understood that the process of artificial hair coloring relies upon damaging the surface of hair fibers so as to create porosity into which dye may be deposited and the demonstrated damage-repairing effect of the product of example 5 may play a role in repairing this local damage. Importantly, it is understood that because chitosan and alginates are susceptible to oxidation by caustics, it is to be expected that re-coloring of the hair would not be impeded by the repairs, which would likely be undone by the chemistry of hair coloring.

### Example 20. OMC/Argan Encapsulated Microemulsion Further Comprising a Peptide

A 10 g aliquot of OMC was combined with 1 gram of argan oil, and this mixture was then combined with 89 g P9. The resulting clear product was then diluted with 300 g of a 0.01% solution of sodium alginate at 50° C., further comprising 0.001% dilution of a pentapeptide, Cysteine-Cysteine-Arginine-Proline-Serine. The resulting product was a water-clear microemulsion similar to others described herein. To this mixture finally was added 100 g of a 0.01% solution of chitosan. The material thus formed was stable to freezing and thawing, and in storage at 45 C for at least one month, remaining water-clear. This product was entirely water-dilutable and self-dispersing.

It will be obvious to one with an ordinary knowledge of Lewis acid and Lewis base salt formation, that any peptide, amino acid sequence, protein, or nucleic acid or polymer thereof bearing at least one charged group can potentially usefully be incorporated into the present disclosure.

### Example 21. Foaming OMC/Argan Cleansing Product

A 1 g aliquot of hydroxystearic acid was dissolved in 44 g OMC, by heating in a boiling water bath and stirring at 300 RPM. To this solution was added 4 grams argan oil, 200 g P9, and 700 g 0.01% sodium alginate. The mixture was heated to 50° C. while stirring at 350 RPM until a clear microemulsion was formed, and then cooled to about 25° C. To the microemulsion product was added 50 g of 0.01% chitosan. The resultant product produced a stable, rich foam when dispensed through an ordinary manual self-foamer dispensing pump. On application to skin, fingernails, and hair, the foamed product provided non-irritating cleansing and pleasant feel, and after rinsing, left detectable OMC deposited onto these surfaces. The product was further useful as a low-irritancy and lubricious shaving foam.

### Example 22: Foaming Argan Protective Barrier Product

A 10 g aliquot of argan oil was combined with 40 g P9, stirring at 300 RPM. To this solution was added 200 g water at 50° C. The mixture was stirred at 350 RPM while cooling to ambient temperature until a clear microemulsion was formed, and then cooled to about 25° C. The resultant product was combined with an additional 200 g of a 1% solution of sodium alginate and aloe vera powder available commercially as ESP A+ gel powder (ESP). The finished composition produced a stable, rich foam when dispensed through an ordinary manual self-foamer dispensing pump. On application to skin, fingernails, and hair, the foamed product provided non-irritating cleansing and pleasant feel. Remarkably, if the product was applied to skin and dried without rinsing, a line drawn in permanent marker over the treated skin could be readily removed by gentle rubbing with water, presumably due to reformation of the microemulsion. If such a line was extended to untreated skin, the mark on the untreated area was indelible and lasted for several days even with vigorous soap and water washing.

### Example 23: Foaming Argan Shaving Product

The product of Example 22 was dispensed through a self-foamer pump and applied to unshaven beard stubble of 1, 2, and 3 days growth after warming the stubble with a damp cloth at approximately 45° C. Approximately 1 g of product was applied and spread over one half of the to be shaved. The other half of the face was treated with a common shaving foam available at pharmacies throughout the United States. Evaluators reported smoother shaves, negligible irritation, and the absence of nicks, bleeding, or redness after use of the product of the present invention, while the side treated with the commercial product consistently produced skin irritation and redness after shaving, and increased nicks and general discomfort by comparison. The product of the present invention was found to provide benefit as a low-irritancy and lubricious shaving foam.

### Example 24. Stain Removal by Microemulsion

Model stains were produced on samples of woven cotton tee-shirt material by application of olive oil and soy oil, as representative food oil stains. These model stains were treated with 10% solutions in water of P9 or Genopol LA 070 (laureth-7, Clariant, Charlotte, N.C.), or dilute microemulsions comprising 10% P9 or Genopol LA 070 respectively, further comprising 1% CTG or 2% lemon oil and 0.5% CTG. The solutions were evaporated to dryness and then rinsed in 40° C. water. After drying the treated stains were inspected to evaluate stain removal. The microemulsions were more effective at removing the oil stains than the solutions of surfactant alone, and no oil residue was visible in the areas treated with the microemulsions.

### Example 25. Transmission Electron Micrograph (TEM) of Charged Polymer Complex

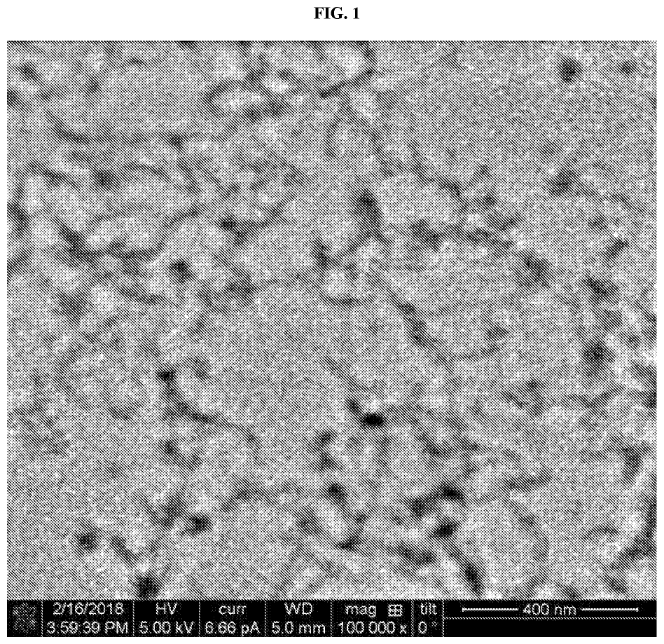

A charged polymer complex microemulsion prepared according to the present invention was imaged by TEM (FIG. 1). This micrograph shows not only discrete points associated with the microemulsion dispersion droplets, but an extended, interconnected network of linked strands of charged polymer complex used to form the microcapsules.

The microencapsulated microemulsion depicted in FIG. 1 was produced according to disclosed methods and further diluted to a 1% content of that material. Approximately 10 μL of the material examined was applied to a standard TEM support grid, allowed to air dry for 5 minutes, and then imaged in the electron microscope. The image in FIG. 1 was processed globally to enhance the contrast of the depicted information without any selective editing of the image information. Images were processed using open-source software ImageJ, available through the US National Institutes of Health (NIH) version 2.0.0-rc-65/1.51u, Build 961c5f1b7f, contrast enhancement set to 0.3% saturated pixels.

### Example 26. Transmission Electron Micrograph (TEM) of Microemulsion without Encapsulating Polymers

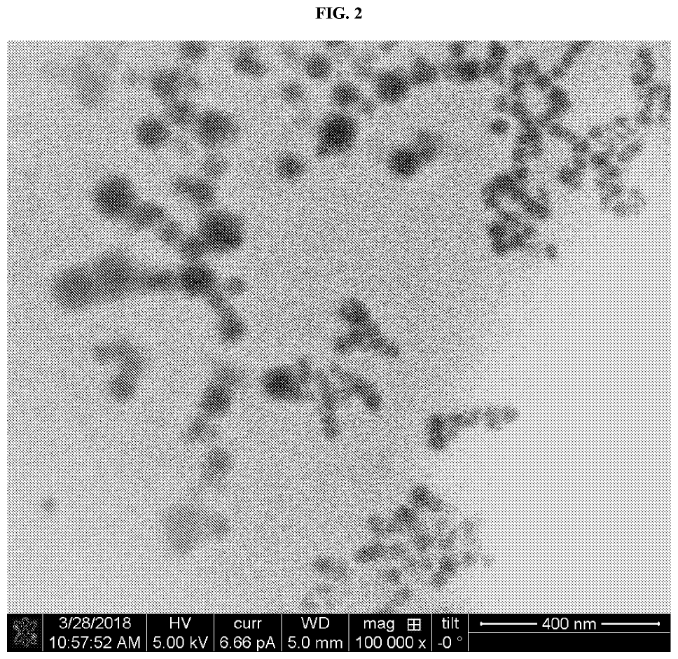

A microemulsion was prepared without any encapsulating polymers according to Example 5, and further diluted to a 1% content of that material (FIG. 2). In this representative image, the absence of any lattice-like network of interconnecting strands may be observed.

Approximately 10 μL of the material examined was applied to a standard TEM support grid, allowed to air dry for 5 minutes, and then imaged in the electron microscope. The image in FIG. 2 was processed globally to enhance the contrast of the depicted information without any selective editing of the image information. Images were processed using open-source software ImageJ, available through the US National Institutes of Health (NIH) version 2.0.0-rc-65/1.51u, Build 961c5f1b7f, contrast enhancement set to 0.3% saturated pixels.

### Example 27. Transmission Electron Micrograph (TEM) of Microencapsulated Microemulsion

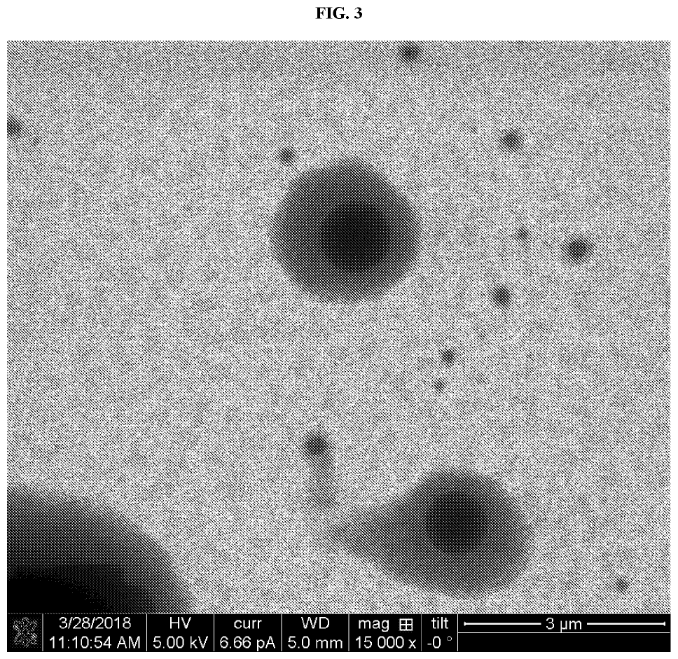

A microencapsulated microemulsion prepared according to the disclosed methods and imaged by TEM (FIG. 3). Some larger droplets are observed, and it is informative to see a cloudlike assemblage of what are understood without limitation to be the associated cationic and anionic polymers that form the encapsulating structures of the composition. The structure of this encapsulating layer is believed without limitation to be more evident in the larger particles due to their size relative to the resolution limit of the microscope. At the lower center of the image, a diffuse association between two particles can be seen, that is understood without limitation to be similar to the associations that form the network visible at higher magnification.

The microencapsulated microemulsion depicted in FIG. 3 was produced according to Example 9, and further diluted to a 1% content of that material. Approximately 10 μL of the material examined was applied to a standard TEM support grid, allowed to air dry for 5 minutes, and then imaged in the electron microscope. The image in FIG. 3 was processed globally to enhance the contrast of the depicted information without any selective editing of the image information. Images were processed using open-source software ImageJ, available through the US National Institutes of Health (NIH) version 2.0.0-rc-65/1.51u, Build 961c5f1b7f, contrast enhancement set to 0.3% saturated pixels.

### REFERENCES

1. Jeirani, Z., et al. “Formulation, optimization and application of triglyceride microemulsion in enhanced oil recovery.” Industrial crops and products 43 (2013): 6-14.
2. Parris, N., R. F. Joubran, and D. P. Lu. “Triglyceride microemulsions: Effect of nonionic surfactants and the nature of the oil.” Journal of agricultural and food chemistry 42.6 (1994): 1295-1299.
3. Szekeres, Erika, Edgar J. Acosta, And James F. Faller. “Microemulsions of triglyceride-based oils: the effect of co-oil and salinity on phase diagrams.” J. Cosmet. Sci 55 (2006): 309-325.
4. Lopes, Luciana B. “Overcoming the cutaneous barrier with microemulsions.” Pharmaceutics 6.1 (2014): 52-77.
5. Heuschkel, Sandra, Alexandra Goebel, and Reinhard H H Neubert. “Microemulsions—modern colloidal carrier for dermal and transdermal drug delivery.” Journal of Pharmaceutical Sciences 97.2 (2008): 603-631.
6. Salager, Jean-Louis, Alain Graciaa, and Jean Lachaise. “Improving solubilization in microemulsions with additives. Part III: lipophilic linker optimization.” Journal of Surfactants and Detergents 1.3 (1998): 403-406.
7. von Corswant, Christian, and Olle Söderman. “Effect of adding isopropyl myristate to microemulsions based on soybean phosphatidylcholine and triglycerides.” Langmuir 14.13 (1998): 3506-3511.
8. Sottmann, Thomas, Cosima Stubenrauch, and C. Stubenrauch. Microemulsions: background, new concepts, applications, perspectives. Chichester: John Wiley & Sons, 2009.
9. WO/2016/127022
10. CN 104382888
11. U.S. Ser. No. 09/259,389
12. U.S. Ser. No. 08/158,134
13. US 2001005337
14. US 20060165739
15. U.S. Pat. No. 8,039,015
16. U.S. Pat. No. 9,744,106

1. A charged polymer complex microemulsion comprising:
(a) a substantially water-immiscible oil phase;
(b) an aqueous phase;
(c) a surfactant; and
(d) a charged polymer complex.

2. The microemulsion of claim 1, wherein the charged polymer complex comprises: one or more amphiphilic anionic polymers, one or more cationic charged polymer, or a combination thereof.

3. The microemulsion of claim 1, wherein the microemulsion is optically transparent.

4. The microemulsion of claim 1, wherein the microemulsion remains optically transparent upon dilution.

5. The microemulsion of claim 1, wherein the microemulsion does not comprise a co-surfactant.

6. The microemulsion of claim 1, wherein the microemulsion does not comprise a metal halide salt.

7. The microemulsion of claim 1, wherein the microemulsion does not comprise a hydrotope.

8.-56. (canceled)

57. A personal care composition comprising the microemulsion of claim 1.

58.-66. (canceled)

67. A method of treating a surface, the method comprising applying the microemulsion of claim 1 to the surface, thereby treating the surface.

68.-73. (canceled)

74. A method of treating hair, the method comprising applying the personal care composition of claim 57 to the hair, thereby treating the hair.

75.-81. (canceled)

82. A method of treating an internal or external surface of a subject, the method comprising applying the personal care composition of claim 57 to the internal or external surface of the subject, thereby treating the internal or external surface.

83.-88. (canceled)

89. A method of laundry care, the method comprising applying the microemulsion of claim 1 to laundry.

90.-91. (canceled)

92. A method of preparing an encapsulated microemulsion, the method comprising admixing:
(a) a substantially water-immiscible oil phase;
(b) an aqueous phase;
(c) a surfactant;
(d) one or more amphiphilic anionic charged polymers; and
(e) one or more amphiphilic cationic charged polymers.

93.-99. (canceled)

100. A composition comprising an oil and a surfactant, wherein:
the oil has an octanol/water partition coefficient (log Kow) of less than 10;
the surfactant HLB is greater than 10;
the composition is substantially free of a co-surfactant, a metal halide salt, a hydrotrope, or a combination of two more thereof;
the composition is optically transparent; and
the composition remains optically transparent upon dilution.

101.-145. (canceled)

146. A personal care composition comprising the composition of claim 100.

147.-153. (canceled)

154. A method of treating a surface, the method comprising applying the composition of claim 100 to the surface, thereby treating the surface.

155.-160. (canceled)

161. A method of treating hair, the method comprising applying the personal care composition of claim 146 to the hair, thereby treating the hair.

162.-167. (canceled)

168. A method of treating an internal or external surface of a subject, the method comprising applying the personal care composition of claim 146 to the internal or external surface of the subject, thereby treating the internal or external surface.

169.-174. (canceled)

175. A method of laundry care, the method comprising applying the composition of claim 100 to laundry.

176.-178. (canceled)

179. A method of preparing the composition of claim 100, the method comprising admixing the surfactant and the oil to provide a mixture.

180.-181. (canceled)


SEQUENCE LISTING (1 sequence)

SEQ ID NO: 1 - protein, 5 aa, Artificial Sequence
  Synthetic
CCRPS